SEQUENCE LISTING (19 sequences)

SEQ ID NO: 1 - DNA, 22 bp, Artificial Sequence
  primer for S. mutans
tcgcgaaaaagataaacaaaca

SEQ ID NO: 2 - DNA, 20 bp, Artificial Sequence
  primer for S. mutans
gccccttcacagttggttag

SEQ ID NO: 3 - DNA, 35 bp, Artificial Sequence
  primer for S. mutans
aaatagattaagacgtagaaaagttaatgggttct

SEQ ID NO: 4 - DNA, 25 bp, Artificial Sequence
  primer for S. mutans
gttgtgatgttatggaggacgagat

SEQ ID NO: 5 - DNA, 17 bp, Artificial Sequence
  oligonucleotide probe for S. mutans
aagcgccacattaacca

SEQ ID NO: 6 - DNA, 24 bp, Artificial Sequence
  primer for S. sanguinis
cggttgttgagtgggagagatttt

SEQ ID NO: 7 - DNA, 25 bp, Artificial Sequence
  primer for S. sanguinis
ccaaggcaatgctaaacaagagaat

SEQ ID NO: 8 - DNA, 15 bp, Artificial Sequence
  oligonucleotide probe for S. sanguinis
atgcgatgaaaattc

SEQ ID NO: 9 - DNA, 479 bp, Streptococcus mutans
tcgcgaaaaagataaacaaacagttgagatccaattaactaaaacaagtcaagatttgaatcattaagaatggacaactatacaatacaacaataagtctgggacaaccataatcccagtcttatttttagataaaacagcagctctttataaatgataactgatagtcaatacaaatcaaatagattaagacgtagaaaagttaatgggttctaagcgccacattaaccagtgacagctagtatagatgttctgcctagcccacagtactagttcgttgtcaaaacatactgatctactgatctcgtcctccataacatcacaactgcctacaatttacaaaacaatacttggagtacggcaagttcaaaaagtccgagaaattctttgaggtgagatatgaagacaacttcgttgacttttagtattaggcagataatagagataacctaaatgtctctaaccaactgtgaaggggc

SEQ ID NO: 10 - DNA, 468 bp, Streptococcus mutans
tcgcgaaaaagataaacaaacagttgagatccaattaactaaaacaagtcaagatttgaatcattaagaatggacaactatacaatacaacaataagtctgggacaaccataatcccagtcttatttttagataaaacagcagctctttataaatgataactgatagtcaatacaaatcaaatagattaagacgtagaaaagttaatgggttctaagcgccacattaaccagtgacagctagtatagatgttctgcctagcccacagtactagttcgttgtcaaatactgatctcgtcctccataacatcacaactgcctacaatttacaaaacaatacttggagtacggcaagttcaaaaagtccgagaaattctttgaggtgagatatgaagacaacttcgttgacttttagtattaggcagataatagagataacctaaatgtctctaaccaactgtgaaggggc

SEQ ID NO: 11 - DNA, 479 bp, Streptococcus mutans
tcgcgaaaaagataaacaaacagttgagatccaattaactaaaacaagtcaagatttgaatcattaagaatggacaactatacaatacaacaataagtctgggacaaccataatcccagtcttatttttagataaaacagcagctctttataaatgataactgatagtcaatacaaatcaaatagattaagacgtagaaaagttaatgggttctaagcgccacattaaccagtgacagctagtatagatgttctgcctagcccacagtactagttcgttgtcaaaacatactgatctactgatctcgtcctccataacatcacaactgcctacaatttacaaaacaatacttggagtacggcaagttcaaaaagtccgagaaattctttgaggtgagatatgaagacaacttcgttgacttttagtattaggcagataatagagataacctaaatatctctaaccaactgtgaaggggc

SEQ ID NO: 12 - DNA, 478 bp, Streptococcus mutans
tcgcgaaaaagataaacaaacagttgagatccaattaactaaaacaagtcaagatttgaatcattaagaatggacaactatacaatacaacaataagtctgggacaaccataatcccagtcttatttttagataaaacagcagctctttataaatgataactgatagtcaatacaaatcaaatagattaagacgtagaaaagttaatgggttctaagcgccacattaaccagtgacagctagtatagatgttctgcctagccacagtactagttcgttgtcaaaacatactgatctactgatctcgtcctccataacatcacaactacctacaatttacaaaacaatacttggagtacggcaagttcaaaaagtccgagaaattctttgaggtgagatatgaagacaacttcgttgacttttagtattaggcagataatagagataacctaaatgtctctaaccaactgtgaaggggc

SEQ ID NO: 13 - DNA, 479 bp, Streptococcus mutans
tcgcgaaaaagataaacaaacagttgagatccaattaactaaaacaagtcaagatttgaatcattaagaacggacaactatacaatacaacaataagtctgggacaaccataatcccagtcttatttttagataaaacagcagctctttataaataataactgatagtcaatacaaatcaaatagattaagacgtagaaaagttaatgggttctaagcgccacattaaccagtgacagctagtatagatgttctgcctagcccacagtactagttcgttgtcaaaacatactgatctactgatctcgtcctccataacatcacaactgcctacaatttacaaaacaatacttggagtacggcaagttcaaaaagtccgagaaattctttgaggtgagatatgaagacaacttcgttgacttttagtattaggcagataatagagataacctaaatgtctctaaccaactgtgaaggggc

SEQ ID NO: 14 - DNA, 468 bp, Streptococcus mutans
tcgcgaaaaagataaacaaacagttgagatccaattaactaaaacaagtcaagatttgaatcattaagaatggacaactatacaatacaacaataagtctgggacaaccataatcccagtcttatttttagataaaacagcagctctttataaatgataactgatagtcaatacaaatcaaatagattaagacgtagaaaagttaatgggttctaagcgccacattaaccagtgacagctagtatagatgttctgcctagcccacagtactagttcgttgtcaaatactgatctcgtcctccataacatcacaactgcctacaatttacaaaacaatacttggagtacggcaagttcaaaaagtccgagaaattctttgaggtgaaatatgaagacaacttcgttgacttttagtattaagcagataatagagataacctaaatgtctctaaccaactgtgaaggggc

SEQ ID NO: 15 - DNA, 468 bp, Streptococcus mutans; identical to SEQ ID NO: 14

SEQ ID NO: 16 - DNA, 479 bp, Streptococcus mutans
tcgcgaaaaagataaacaaacagttgagatccaattaactaaaacaagtcaagatttgaatcattaagaatggacaactatacaatacaacaataagtctgggacaaccataatcccagtcttatttttagataaaacagcagctctttataaatgataactgatagtcaatacaaatcaaatagattaagacgtagaaaagttaatgggttctaagcgccacattaaccagtgacagctagtatagatgttctgcctagcccacagtactagttcgttgtcaaaacatactgatctactgatctcgtcctccataacatcacaactacctacaatttacaaaacaatacttggagtacggcaagttcaaaaagtccgagaaattctttgaggtgaaatatgaagacaacttcgttgacttttagtattaagcagataatagagataacctaaatgtctctaaccaactgtgaaggggc

SEQ ID NO: 17 - DNA, 475 bp, Streptococcus sanguinis
gaagccattttgcctagattgatggaagtagttcatcatctggtaatatgctttcatgaatacaaactgaatacgcaaaactgaggcagaactgtctcagttttcttttccaaagaaaaaggggggactatttagttcgtccctgactggagtcttgtggtataataaaagttatggtagaagttgtttttaaattatgcagtcatttgctctttattttctttgcctttcatctgctgtcaacggttgttgagtgggagagatttttcaaggttaatgccgataatgcgatgaaaattcgctttttgattctcttgtttagcattgccttgggctatttagtcagccttttcttcattggaatttacgatatgagccgggcgatttttaatggtactttataaggtcatatttttgattttatggtatgatagtatggatgattttaaaaatttagagtaaggaatcggtatgg

SEQ ID NO: 18 - DNA, 25 bp, Artificial Sequence
  primer for S. sanguinis
gaagccattttgcctagattgatgg

SEQ ID NO: 19 - DNA, 26 bp, Artificial Sequence
  primer for S. sanguinis
ccataccgattccttactctaaattt

(19) United States
(12) Patent Application Publication
(10) Pub. No.: US 2009/0305252 A1
(43) Pub. Date: Dec. 10, 2009

(54) Methods and species-specific primers for detection and quantification of Streptococcus mutans and Streptococcus sanguinis in mixed bacterial samples

(76) Inventors: Yihong Li, New York, NY (US); Page W. Caufield, New York, NY (US); Deepak Saxena, New York, NY (US); Zhou Chen, New York, NY (US)

Correspondence Address: KLAUBER & JACKSON, 411 HACKENSACK AVENUE, HACKENSACK, NJ, 07601, US

(21) Appl. No.: 12/156,955

(22) Filed: Jun. 4, 2008

Related U.S. Application Data
(60) Provisional application No. 60/933,234, filed on Jun. 5, 2007.

(51) Int. Cl. C12Q 1/68; C07H 21/00

(52) U.S. Cl. 435/6; 536/24.33

(57) ABSTRACT

Dental caries is a polymicrobial infectious disease. Of the hundreds of bacteria present in the biofilms coating teeth, the Streptococcus mutans (S. mutans) remain strongly linked to caries and dental disease. Streptococcus sanguinis (S. sanguinis) may serve a protective or antagonistic role against the cariogenic bacterium S. mutans. In the present invention, exemplary sets of species-specific PCR primers are provided for the identification and quantification of S. mutans and of S. sanguinis in clinical samples, including the simultaneous and sensitive analysis of both bacterial species. Assays, kits and methods for determining the presence and amount of S. mutans and/or S. sanguinis are provided. Oligonucleotide probes and primers for use in the assays, kits and methods are described. Assays and methods for determining and evaluating an individual's oral bacteria, risk for caries, and effects of prevention and treatment modalities, are provided.

### CROSS REFERENCE TO RELATED APPLICATIONS

The present application claims priority of co-pending provisional application U.S. Ser. No. 60/933,234, filed on Jun. 5, 2007, the disclosure of which is incorporated by reference herein in its entirety. Applicants claim the benefits of such application under 35 U.S.C. §119(e).

### GOVERNMENTAL SUPPORT

The research leading to the present invention was supported, at least in part, by a grant from the National Institutes of Dental and Craniofacial Research, National Institutes of Health, Grant No. DE015706. Accordingly, the Government has certain rights in the invention.

### FIELD OF THE INVENTION

The present invention relates generally to assessment of oral bacteria species associated with dental caries, including Streptococcus mutans (S. mutans) and Streptococcus sanguinis (S. sanguinis). The invention also relates to assays, kits and methods for determining the presence and amount of S. mutans and/or S. sanguinis. Exemplary sets of species-specific PCR primers and probes are provided for the identification and quantification of S. mutans and of S. sanguinis in clinical samples.

### BACKGROUND OF THE INVENTION

Dental caries is a microbial infectious disease. Of the hundreds of bacteria present in the biofilms coating teeth, the Streptococcus mutans remain strongly linked to caries in terms of its metabolic, ecological, and epidemiological associations. A high concentration and early acquisition of S. mutans in the oral cavity are indicators of a high risk for caries. Streptococcus sanguinis (formerly S. sanguis), another key member of the indigenous oral biota, is one of the most prevalent members of the oral streptococci, and is correlated with dental health. S. sanguinis may serve a protective or antagonistic role against the cariogenic bacterium S. mutans (Caufield, P. W. et al. (2000) Infect. Immun. 68:4018-4023; Loesche, W. J. and Syed, S. A. (1973) Caries Res. 7:201-216; Loesche, W. J. et al (1973) Arch. Oral Biol. 18:571-575). Based on conventional culture methods, it has been suggested that S. mutans/S. sanguinis ratio may serve as a caries risk indicator.

To the extent that certain environmental or biological factors may trigger a disruption in the balance of oral bacterial species, leading to microbial diseases, the balance and interaction between S. sanguinis and S. mutans has been studied (Kreth, J. et al (2005) J Bact 187(21):7193-7203). Because the modulation of S. sanguinis and S. mutans colonization may naturally influence the development and progression of dental caries, a rapid, reliable and quantitative assay for testing these bacterial species in a clinical sample or a subject's oral cavity is of significant benefit, both as a tool in development of therapies and for analysis and active use in monitoring, prophylaxis and therapy.

Conventionally, studies of oral bacterial species, including S. mutans have relied heavily upon cultivation to identify and characterize S. mutans in the oral cavity. The major limitations of culture methods include: a limited threshold of detection of S. mutans in clinical samples; an inconsistent morphology of S. mutans depending on the culture medium used; and its high cost and labor intensiveness. In addition, S. mutans cultivation requires viable samples, making its application in field studies impractical.

Given the significant and important relevance of dental caries as an infectious disease in dental care and clinical management of dental patients, the ability to monitor and predict the existence and extent of S. mutans, in clinical and epidemiological assessment and studies is paramount. A knowledge of, and the ability to readily and rapidly predict and assess the presence and amount of S. mutans in a patient's oral cavity or saliva, particularly a young patient's, is therefore needed. This is also useful and important in determining the response of S. mutans, to dental and oral therapeutic and care intervention.

A number of specific probes have been reported for species-specific genes associated with virulence in S. mutans, such as glucosyltransferases, fructosyltransferases, dextranase, glucan-binding protein B, cell surface protein, the phosphoenolpyruvate-dependent sucrose phosphotransferase system, and protein antigen. Although these primers work well with pure S. mutans cultures, however, they also show some cross-reactions with other bacterial species in the oral cavity and many of them have not been validated against mixed clinical specimens.

In the present invention, the inventors have designed and validated exemplary sets of species-specific PCR primers for the identification and quantification of S. mutans and of S. sanguinis in clinical samples, including for simultaneous analysis of both bacterial species. These exemplary sets of PCR primers are highly specific and sensitive for identification of S. mutans in either pure culture of S. mutans or in mixed culture (clinical sample). A separate set of PCR primers have been designed for S. sanguinis and are specific for identification of S. sanguinis in pure or mixed culture. PCR identification of S. mutans and/or S. sanguinis using these species-specific primers is reliable and fast. A viable sample is not required; therefore, the assay and method may be used for conducting high-throughput clinical and epidemiological studies.

The citation of references herein shall not be construed as an admission that such is prior art to the present invention.

### SUMMARY OF THE INVENTION

The present invention provides an approach, method, and assays to detect bacteria in dental samples and determine or predict dental caries and cavities in an individual, particularly in children. The approach involves the detection, particularly quantitative detection, of bacteria which are positively and/or negatively correlated with or predictive of caries and dental disease.

In a particular aspect S. mutans is correlated positively with dental caries and S. sanguinis has a negative correlation with dental caries. The present invention demonstrates that primer set(s) directed against Sm479F/Sm479R can accurately and rapidly identify and quantify S. mutans in clinical samples or in subjects. These species-specific primers and probes may be used for conducting high-throughput epidemiological studies, monitoring, and assessment of S. mutans infection and the response of S. mutans to prophylaxis and therapy. Similarly, sets of S. sanguinis-specific primers have been developed to identify and quantify S. sanguinis in clinical samples. The S. sanguinis primers and primer set(s) are directed against the Sa475F/Sa475R (SSA-2) target region. These probes are useful and applicable in real time quantitative PCR assays and methods. Exemplary direct product readout assays have been developed. Collectively and in combination, the specific and sensitive S. mutans and S. sanguinis probes enable the simultaneous measurement of S. mutans and S. sanguinis presence and determination of the S. mutans/S. sanguinis ratios, which will provide clinicians with a valuable diagnostic tool indicating the presence of a cariogenic biota, hence likelihood for developing dental caries.

The present invention extends to diagnostic assays, kits and methods for determining the presence or amount of S. mutans bacterium in a sample or subject. The present invention extends to diagnostic assays, kits and methods for determining the presence or amount of S. sanguinis bacterium in a sample or subject. The present invention further extends to duplex or combination assays, kits and methods for determining the presence or amount of both S. mutans and S. sanguinis bacteria, including the relative amount or ratio of these two bacteria. This is particularly relevant in determining and assessing a subject's risk for dental caries, as well as the cariogenic potential of saliva and dental plaque of an individual. This is also relevant in determining and assessing the bacterial populations' response to treatment and prevention measures and therapies. Thus, methods, kits and assays are provided for determining the caries risk or caries status of an individual based on the relative amounts of S. mutans and S. sanguinis or the S. mutans/S. sanguinis ratio of an individual and in a sample from an individual.

In accordance with the present invention, a diagnostic assay is provided for determining the presence or amount of S. mutans in a sample or subject which comprises

(a) isolating nucleic acid from said sample or subject;
(b) amplifying Sm479F/Sm479R targeted S. mutans sequence or a portion thereof using PCR or other amplification technology; and
(c) determining the presence and amount of the Sm479F/Sm479R targeted PCR product sequence, thereby determining the presence or amount of S. mutans in said sample or subject.

In accordance with the present invention, a diagnostic assay is provided for determining the presence or amount of S. mutans in a sample or subject which comprises

(a) isolating nucleic acid from said sample or subject;
(b) amplifying an Sm479F/Sm479R targeted S. mutans sequence PCR fragment from said nucleic acid; and
(c) detecting and quantitating the amplified Sm479F/Sm479R targeted S. mutans sequence or portion thereof obtained in step (b), thereby determining the presence or amount of S. mutans in said sample or subject.

In accordance with the present invention, a diagnostic assay is provided for determining the presence or amount of S. sanguinis in a sample or subject which comprises

(a) isolating nucleic acid from said sample or subject;
(b) amplifying Sa475F/Sa475R targeted S. sanguinis sequence or a portion thereof using PCR or other amplification technology; and
(c) determining the presence and amount of the S. sanguinis Sa475F/Sa475R targeted PCR product sequence, thereby determining the presence or amount of S. sanguinis in said sample or subject.

In accordance with the present invention, a diagnostic assay is provided for determining the presence or amount of S. sanguinis in a sample or subject which comprises

(a) isolating nucleic acid from said sample or subject;
(b) amplifying Sa475F/Sa475R targeted S. sanguinis sequence PCR fragment from said nucleic acid; and
(c) detecting and quantitating the amplified Sa475F/Sa475R targeted S. sanguinis sequence or portion thereof obtained in step (b), thereby determining the presence or amount of S. sanguinis in said sample or subject.

In accordance with the present invention, a diagnostic assay is provided for simultaneously determining the presence or amount of S. mutans and S. sanguinis in a sample or subject which comprises

(a) isolating nucleic acid from said sample or subject;
(b) amplifying both Sm479F/Sm479R targeted S. mutans sequence PCR fragment and Sa475F/Sa475R S. sanguinis sequence PCR fragment from said nucleic acid; and
(c) detecting and quantitating both of the amplified portion of Sm479F/Sm479R targeted S. mutans sequence and the amplified portion of Sa475F/Sa475R targeted S. sanguinis sequence obtained in step (b), thereby determining the presence or amount of both S. mutans and S. sanguinis in said sample or subject.

In an aspect, the present invention provides a diagnostic assay for simultaneously determining the presence or amount of S. mutans and S. sanguinis in a sample or subject which comprises

(a) isolating nucleic acid from said sample or subject;
(b) amplifying both Sm479F/Sm479R targeted S. mutans sequence PCR fragment and Sa475F/Sa475R targeted S. sanguinis sequence PCR fragment from said nucleic acid using a set of primers, wherein said set of primers contains primer pair X and Y and primer pair A and B; wherein

(i) the X and Y primer pair are complementary to a portion of the Sm479F/Sm479R targeted S. mutans sequence;

(ii) the A and B primer pair are complementary to a portion of the Sa475F/Sa475R targeted S. sanguinis sequence;

(c) amplifying both the sequence in between primers X and Y and the sequence in between primers A and B, thereby obtaining two distinct amplified fragments; and
(d) detecting and quantitating the amplified fragments obtained in step (c), thereby determining the presence or amount of S. mutans and S. sanguinis in said sample or subject.

In a particular example, the present invention extends to a diagnostic assay, wherein primer X has the sequence corresponding to SEQ ID NO: 3, or a fragment thereof which is at least ten bases long, primer Y has the sequence corresponding to SEQ ID NO: 4, or a fragment thereof which is at least ten bases long, primer A has the sequence corresponding to SEQ ID NO: 6, or a fragment thereof which is at least ten bases long, primer Y has the sequence corresponding to SEQ ID NO: 7. In a further example, the present invention extends to a diagnostic assay, wherein primer X has the sequence corresponding to SEQ ID NO: 1, or a fragment thereof which is at least ten bases long, and primer Y has the sequence corresponding to SEQ ID NO: 2, or a fragment thereof which is at least ten bases long. In a further example, the present invention extends to a diagnostic assay, wherein primer A has the sequence corresponding to SEQ ID NO: 18, or a fragment thereof which is at least ten bases long, and primer B has the sequence corresponding to SEQ ID NO: 19, or a fragment thereof which is at least ten bases long. In a particular example, the present invention extends to a diagnostic assay, wherein primer X has the sequence corresponding to SEQ ID NO: 1, or a fragment thereof which is at least ten bases long, primer Y has the sequence corresponding to SEQ ID NO: 2, or a fragment thereof which is at least ten bases long, primer A has the sequence corresponding to SEQ ID NO: 18, or a fragment thereof which is at least ten bases long, primer Y has the sequence corresponding to SEQ ID NO: 19.

The invention provides a method for determining the caries risk or caries status of an individual based on the relative amounts of S. mutans and S. sanguinis or the S. mutans/S. sanguinis ratio of an individual and in a sample from an individual which comprises

(a) isolating nucleic acid from said sample or subject;
(b) amplifying both Sm479F/Sm479R targeted S. mutans sequence PCR fragment and Sa475F/Sa475R targeted S. sanguinis sequence PCR fragment from said nucleic acid; and
(c) detecting and quantitating both of the amplified portion of Sm479F/Sm479R targeted S. mutans sequence and the amplified portion of Sa475F/Sa475R targeted S. sanguinis sequence obtained in step (b), thereby determining the relative amounts of S. mutans and S. sanguinis or the S. mutans/S. sanguinis ratio in said sample or subject, whereby an increased or relatively elevated S. mutans/S. sanguinis ratio indicates active caries or caries risk in said individual.

In accordance with the present invention, a diagnostic assay is provided for determining the presence or amount of S. mutans in a sample or subject which comprises

(a) isolating nucleic acid from said sample or subject;
(b) amplifying Sm479F/Sm479R targeted S. mutans sequence or a portion thereof using PCR or other amplification technology in the presence of a fluorogenic probe; and
(c) determining the presence and amount of the Sm479F/Sm479R targeted PCR product sequence, wherein the amount of fluorescence is indicative of the presence and amount of PCR product, thereby determining the presence or amount of S. mutans in said sample or subject.

In a further aspect of the present invention, a diagnostic assay is provided for determining the presence or amount of S. sanguinis in a sample or subject which comprises

(a) isolating nucleic acid from said sample or subject;
(b) amplifying Sa475F/Sa475R targeted S. sanguinis sequence or a portion thereof using PCR or other amplification technology in the presence of a fluorogenic probe; and
(c) determining the presence and amount of the Sa475F/Sa475R targeted PCR product sequence, wherein the amount of fluorescence is indicative of the presence and amount of PCR product, thereby determining the presence or amount of S. sanguinis in said sample or subject.

In a still further aspect, a diagnostic assay is provided for simultaneously determining the presence or amount of S. mutans and S. sanguinis in a sample or subject which comprises

(a) isolating nucleic acid from said sample or subject;
(b) amplifying from said nucleic acid both Sm479F/Sm479R targeted S. mutans sequence PCR fragment in the presence of an S. mutans product specific fluorogenic probe, and Sa475F/Sa475R targeted S. sanguinis sequence PCR fragment in the presence of an S. sanguinis product specific fluorogenic probe, wherein the fluorogenic probes have distinct fluorophores; and
(c) determining the presence and amount of both the Sm479F/Sm479R PCR product sequence and the Sa475F/Sa475R S. sanguinis sequence PCR fragment, wherein the amount of fluorescence of each fluorophore is indicative of the presence and amount of PCR product, thereby determining the presence or amount of both S. mutans and S. sanguinis in said sample or subject.

In one embodiment, the above assays utilize a set of Sm479F/Sm479R S. mutans primers selected from SEQ ID NO: 1 and 2 or SEQ ID NO: 3 and 4 and, optionally, a fluorogenic probe sequence of SEQ ID NO: 5. In a further embodiment, the above assays utilize a set of Sa475F/Sa475R S. sanguinis primers selected from SEQ ID NO: 6 and 7 and, optionally, a fluorogenic probe sequence of SEQ ID NO: 8.

In a further aspect, the present invention provides a test kit for determining the presence or amount of S. mutans and/or S. sanguinis in a sample or subject, comprising:

(a) a predetermined amount of a first PCR primer set which amplifies Sm479F/Sm479R targeted S. mutans sequence or a portion thereof;
(b) a predetermined amount of a second PCR primer set which amplifies Sa475F/Sa475R targeted S. sanguinis sequence or a portion thereof;
(c) other reagents, optionally including a fluorogenic probe specific for the amplified Sm479F/Sm479R targeted S. mutans PCR product and a distinct fluorogenic probe specific for the amplified Sa475F/Sa475R targeted S. sanguinis PCR product; and
(d) directions for use of said kit.

In a particular embodiment, the first PCR primer set in a test kit of the present invention has sequences corresponding to SEQ ID NO: 1 and SEQ ID NO: 2, or SEQ ID NO: 3 and SEQ ID NO:4, or fragments thereof which are at least ten bases long.

In a further particular embodiment, the second PCR primer set in a test kit of the present invention has sequences corresponding to SEQ ID NO: 6 and SEQ ID NO: 7, or SEQ ID NO: 18 and SEQ ID NO: 19, or fragments thereof which are at least ten bases long.

In a still further particular embodiment, the S. mutans Sm479F/Sm479R product fluorogenic probe has a sequence corresponding to SEQ ID NO: 5, or a fragment thereof which is at least ten bases long. In another particular embodiment, the S. sanguinis Sa475F/Sa475R product fluorogenic probe has a sequence corresponding to SEQ ID NO: 8, or a fragment thereof which is at least ten bases long.

The invention also relates to an isolated oligonucleotide primer having a sequence selected from SEQ ID NO: 1, 2, 3, 4, 6 or 7, or a fragment thereof which is at least ten bases long. The isolated oligonucleotide primer may have a sequence selected from SEQ ID NO: 18 or 19, or a fragment thereof which is at least ten bases long. The invention provides a composition of a primer pair and probe set comprising the sequences SEQ ID NO: 3, 4 and 5 in combination suitable for amplification and detection of S. mutans in a sample. The invention provides a composition of a primer pair and probe set comprising the sequences SEQ ID NO: 6, 7 and 8 in combination suitable for amplification and detection of S. sanguinis in a sample. In a further embodiment, the invention includes a composition of primer pairs and probes in combination, suitable for simultaneous amplification and detection of S. mutans and S. sanguinis in a sample, comprising the sequences SEQ ID NO: 3, 4, 5, 6, 7 and 8.

In an aspect of the invention, the fluorophore on the fluorogenic probe(s) may be selected from 6-carboxyfluoroscein (FAM), 2′-chloro-7′-phenyl-1,4-dichloro-6-carboxyfluorescein (VIC), tetrachloro-6-carboxyfluorescein, and hexachloro-6-carboxyflorescein. In a further aspect, the fluorogenic probe(s) further comprise a covalently attached quencher. The quencher may be selected from a non-fluorescent quencher, such as a minor groove binder, and a fluorescent quencher, such as 6-caboxytetramethylrhodamine (TAMRA).

Other objects and advantages will become apparent to those skilled in the art from a review of the following description which proceeds with reference to the following illustrative drawings.

### BRIEF DESCRIPTION OF THE DRAWINGS

FIGS. 1A and 1B depicts the development of species-specific primers for S. mutans. (A) Chromosomal DNA fingerprint profiles of different Streptococcus species after HaeIII restriction enzyme digestion and electrophoresis in a 0.55% agarose gel. Lane 1-5, S. mutans reference strains 10449, KPSK2, Ingbritt, UA159, and OMZ175. Lane 6, S. sobrinus reference strain OMZ65. Lane 7, S. sanguinis reference strain ATCC10556. The unique 14-kb fragment was observed among all S. mutans strains, but not other streptococcus strains. (B) Locations of the Sm479F/R primers. The primers were designed to anneal to sequences within the unique 13,693-bp fragment, which encompasses nt 2021910 to nt 4682 of the UA159 genome (AE014133). The targeted segment comprises a portion of the htrA locus and a part of an intergenic locus of the S. mutans genome. The final size of the PCR amplicon is 479 base pairs.

FIGS. 2A, 2B and 2C depicts evaluation of the species specificity and the limit of detection of the primers by PCR. (A) Gel electrophoresis of PCR products of the reference strains (16 out of a total of 55 are illustrated) of mutans streptococci and other non-mutans streptococci species using the primers Sm479F/Sm479R. The agarose gel shows the PCR-amplified target DNA to be present in the S. mutans type-strains and absent in the non-S. mutans strains tested with a high degree of specificity. The molecular size standard consisting of a 100-bp DNA ladder is shown in the first lane. (B) Detection of S. mutans DNA by PCR using the Sm479F/Sm479R primers against 5-fold serially diluted concentrations of pure UA159 DNA samples. The minimum detectable level was ≧1.6×10⁻² ng. (C) Detection of S. mutans DNA by PCR using the Sm479F/R primers against serially diluted UA159 genomic DNA samples mixed with S. sanguinis (ATCC10556) or S. sobrinus (OMZ65) DNA. The lowest detection level for S. mutans was 0.01 ng.

FIG. 3 depicts evaluation of the specificity Sm479F/Sm479R primers by PCR. DNA amplification was observed from the S. mutans (UA159) strain (Lane 2), but not from human buccal mucosa epithelial cells (hEt) (Lane 3), nor from a human whole blood sample (Lane 4) or the negative control (Lane 5). The results further support the conclusion that the Sm479F/R primers are not only specific for S. mutans, but also do not display cross-reactivity with human DNA samples.

FIG. 4 depicts an alignment of the nucleotide sequences of the Sm479F/Sm479R amplicons from UA159, ATCC25175 (10449), Ingbritt, GS5, LM7, OMZ175 and two randomly selected mixed bacterial samples (25-2 and 25-18) (SEQ ID NOS 9-16, respectively). “*” indicates the residues or nucleotides in that column are identical in all sequences in the alignment. The results demonstrated 98% to 100% identities among different S. mutans serotype strains.

FIG. 5 depicts the nucleotide sequence of the Sa475F/Sa475R targeted (SSA-2) region of S. sanguinis (SEQ ID NO: 17). The SSA-2 region is part of a 1,653 bp fragment from strains of S. sanguinis. The sequence of SSA-2, one of several probe regions tested for S. sanguinis specificity, comprises an intergenic region between the first ORF (uncC gene) and the second ORF (murA gene) of S. sanguinis.

FIGS. 6A, 6B and 6C depict standard curves and clinical samples of S. mutans and S. sanguinis (A and B, respectively) and (C) combined standard curves for both S. mutans and S. sanguinis using duplex real-time qPCR analysis.

FIGS. 7A, 7B and 7C depicts real-time qPCR analysis of S. mutans or S. sanguinis levels. (A) Curve of the fluorescence versus cycle number obtained from the SYBR Green detection of S. sanguinis DNA portion. The line represents the threshold arbitrarily set in the middle of the log fluorescence. (B) Melting curve analysis. Following the final PCR cycle the samples were subjected to a melting curve analysis over the indicted temperature range. (C) Sensitivity and dynamic range of real-time qPCR. Serial dilutions of genomic DNA from S. sanguinis 10556 were amplified using real-time qPCR. The dynamic range for this assay is presented between 10 ng and 10 fg.

FIGS. 8A and 8B depicts a comparison of the cumulative detection rate of S. mutans (A) or S. sanguinis (B) by the culture method and the real-time qPCR method (N=584 samples). The results show a higher positive detection rate for PCR at different ages.

FIG. 9 provides standard curves which show the linear correlations between quantity of standard DNA ( for S. mutans UA159 and ▴ for S. sanguinis 10556) in serial dilutions (10¹˜10⁻⁶ ng) and the C(T) values in a single plate.

FIG. 10 depicts confirmation of the duplex real-time qPCR in 2% agarose gels. The results show the positive detection of S. mutans and S. sanguinis amplicons are 147-bp and 90-bp, respectively. M=50 bp DNA ladder.

FIG. 11 provides linear regression analysis that shows a significant positive correlation (p=0.003) between S. mutans levels and caries severity.

FIG. 12 provides linear regression analysis that shows that a negative trend in S. sanguinis level correlated and caries severity.

FIG. 13 provides linear regression analysis that shows a positive correlation (marginal significance, p=0.062) between the ratio of S. mutans/S. sanguinis and caries severity.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

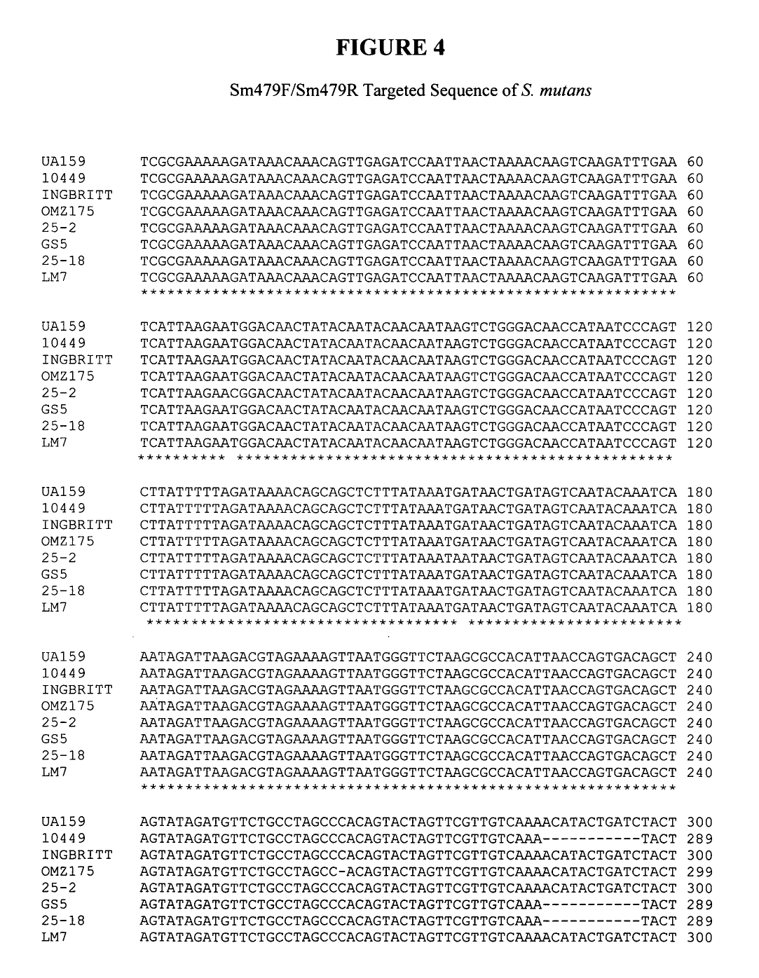

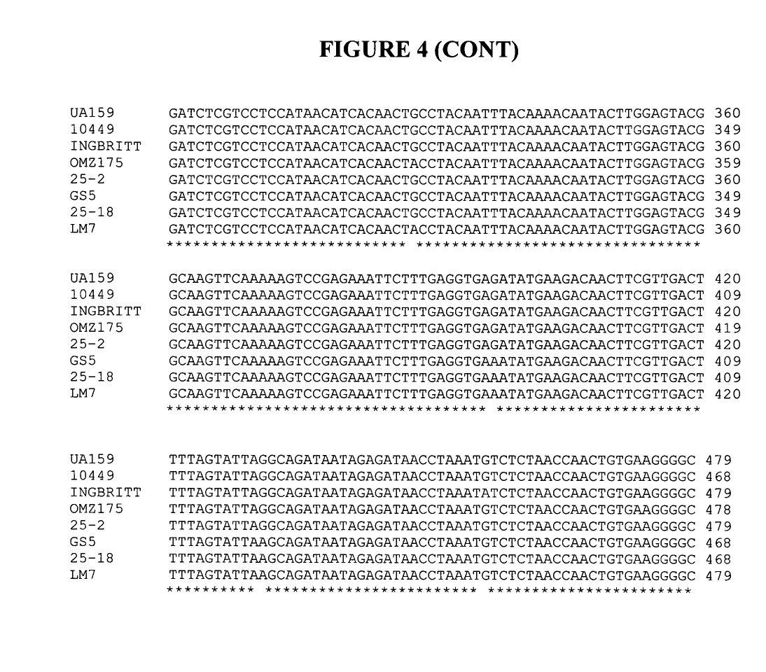

The terms “Sm479”, “Sm479 region”, “Sm479F/R”, “Sm479F/Sm479R”, and “expsm479-F/expsm479-R” and any variants not specifically listed, may be used herein interchangeably, and as used throughout the present application and claims refer to a nucleotide or nucleic acid sequence region of Streptococcus mutans (S. mutans), and extends to those nucleic acids having the nucleotide sequence data described herein and presented in FIG. 4 (SEQ ID NOS: 9-16), and the profile of activities and characteristics set forth herein and in the Claims. Accordingly, nucleic acids and nucleotides displaying substantially equivalent or altered activity or sequence are likewise contemplated. These modifications may be deliberate, for example, such as modifications obtained through site-directed mutagenesis, or may be accidental, such as those obtained through mutations in S. mutans or variant bacteria from any of various sources, cultures or hosts. Also, the terms “Sm479”, “Sm479 region”, “Sm479F/R”, “Sm479F/Sm479R”, and “expsm479-F/expsm479-R” are intended to include within their scope nucleic acids specifically recited herein as well as all substantially homologous analogs and allelic variations.

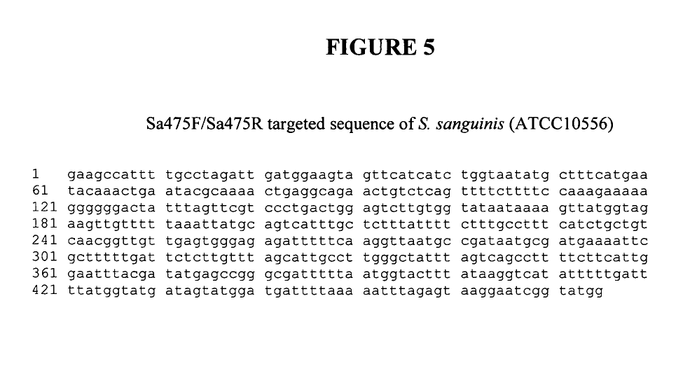

The terms “SSA-2”, “SSA-2 region”, “SSA-2F/R”, “Sa475”, “Sa475F/R”, “Sa475F/Sa475R”, and “expsa475-F/expsa475-R” and any variants not specifically listed, may be used herein interchangeably, and as used throughout the present application and claims refer to a nucleotide or nucleic acid sequence region of Streptococcus sanguinis (S. sanguinis), and extends to those nucleic acids having the nucleotide sequence data described herein and presented in FIG. 5 (SEQ ID NO: 17), and the profile of activities and characteristics set forth herein and in the Claims. Accordingly, nucleic acids and nucleotides displaying substantially equivalent or altered activity or sequence are likewise contemplated. These modifications may be deliberate, for example, such as modifications obtained through site-directed mutagenesis, or may be accidental, such as those obtained through mutations in S. mutans or variant bacteria from any of various sources, cultures or hosts. Also, the terms “SSA-2”, “SSA-2 region”, “SSA-2F/R”, “Sa475”, “Sa475F/R”, “Sa475F/Sa475R”, and “expsa475-F/expsa475-R” are intended to include within their scope nucleic acids specifically recited herein as well as all substantially homologous analogs and allelic variations.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are as follows: Y (Tyr, tyrosine); G (Gly, glycine); F (Phe, phenylalanine); M (Met, methionine); A (Ala, alanine); S (Ser, serine); I (Ile, isoleucine); L (Leu, leucine); T (Thr, threonine); V (Val, valine); P (Pro, proline); K (Lys, lysine); H (His, histidine); Q (Gln, glutamine); E (Glu, glutamic acid); W (Trp, tryptophan); R (Arg, arginine); D (Asp, aspartic acid); N (Asn, asparagine); and C (Cys, cysteine).

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence. A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences and oligonucleotides or primers which are degenerate to the sequences set out herein, including SEQ ID NO: 1, 2, 3, 4, 6, 7, 18, 19, etc., and would encode or code for the same amino acid sequence as a genomic or expressed such sequence, but which are degenerate to the sequences set out herein, including SEQ ID NO: 1, 2, 3, 4, 6, 7, 18, 19, etc. By “degenerate to” is meant that a different three-letter codon is used to specify a particular amino acid. It is well known in the art that the codons can be used interchangeably to code for each specific amino acid, including as set out below:

| | |
|---|---|
| Phenylalanine (Phe or F) | UUU or UUC |
| Leucine (Leu or L) | UUA or UUG or CUU or CUC |
| | or CUA or CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG |
| | or AGU or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG |
| | or AGA or AGG |
| Glycine (Gly or G) | GGU or GGC or GGA or GGG |
| Tryptophan (Trp or W) | UGG |
| Termination codon | UAA (ochre) or UAG (amber) |
| | or UGA (opal) |

It should be understood that the codons specified above are for RNA sequences. The corresponding codons for DNA have a T substituted for U.

Mutations can be made in the sequences set out herein, including SEQ ID NO: 1, 2, 3, 4, 6, 7, 18, 19, etc., including such that a particular codon therein is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. Mutations or alterations in the sequences set out herein, including SEQ ID NO: 1, 2, 3, 4, 6, 7, 18, 19, etc. may reflect population variation or even mutation in the bacterial species. The present invention should be considered to include sequences containing conservative changes, variations and mutations.

Two sequences are “substantially homologous” when at least about 70% of the sequence residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant change in the S phase activity of a target cellular mass, or other feature of pathology such as for example, elevated blood pressure, fever or white cell count as may attend its presence and activity.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

In its broadest aspect, the present invention extends to diagnostic assays, kits and methods for determining the presence or amount of oral bacteria species, including of S. mutans and/or S. sanguinis in a sample or subject. This is particularly relevant in determining and assessing the prevalence of oral bacteria species, particularly of S. mutans and/or S. sanguinis, and their relevance to dental caries, caries risk, and dental disease. It is also relevant and applicable in evaluating or determining appropriate preventive care and therapy in dental patients.

The assays and methods of the present invention broadly and generally include and incorporate the following steps in determining the presence or amount of S. mutans in a sample or subject which comprises

(a) isolating nucleic acid from said sample or subject;
(b) amplifying Sm479F/Sm479R targeted S. mutans sequence or a portion thereof using PCR or other amplification technology; and
(c) determining the presence and amount of the Sm479F/Sm479R targeted PCR product sequence, thereby determining the presence or amount of S. mutans in said sample or subject.

The assays and methods of the present invention broadly and generally include and incorporate the following steps in determining the presence or amount of S. sanguinis in a sample or subject which comprises

(a) isolating nucleic acid from said sample or subject;
(b) amplifying Sa475F/Sa475R targeted S. sanguinis sequence or a portion thereof using PCR or other amplification technology; and
(c) determining the presence and amount of the S. sanguinis Sa475F/Sa475R targeted PCR product sequence, thereby determining the presence or amount of S. sanguinis in said sample or subject. A diagnostic assay is also provided for simultaneously determining the presence or amount of S. mutans and S. sanguinis in a sample or subject which comprises
(a) isolating nucleic acid from said sample or subject;
(b) amplifying both Sm479F/Sm479R targeted S. mutans sequence PCR fragment and Sa475F/Sa475R targeted S. sanguinis sequence PCR fragment from said nucleic acid; and
(c) detecting and quantitating both of the amplified portion of Sm479F/Sm479R targeted S. mutans sequence and the amplified portion of Sa475F/Sa475R targeted S. sanguinis sequence obtained in step (b), thereby determining the presence or amount of both S. mutans and S. sanguinis in said sample or subject.

A method is provided for determining the caries risk or caries status of an individual based on the relative amounts of S. mutans and S. sanguinis or the S. mutans/S. sanguinis ratio of an individual and in a sample from an individual which comprises

(a) isolating nucleic acid from said sample or subject;
(b) amplifying both Sm479F/Sm479R targeted S. mutans sequence PCR fragment and Sa475F/Sa475R targeted S. sanguinis sequence PCR fragment from said nucleic acid; and
(c) detecting and quantitating both of the amplified portion of Sm479F/Sm479R targeted S. mutans sequence and the amplified portion of Sa475F/Sa475R targeted S. sanguinis sequence obtained in step (b), thereby determining the relative amounts of S. mutans and S. sanguinis or the S. mutans/S. sanguinis ratio in said sample or subject, whereby an increased or relatively elevated S. mutans/S. sanguinis ratio indicates active caries or caries risk in said individual.

The term “isolation”, “isolating” or “isolate” as used herein, and as applied to the methods and assays described herein, refers to and encompasses any method or approach known in the art whereby DNA can be obtained, procured, prepared, purified or isolated such that it is suitable for analysis, amplification, restriction enzyme cleavage and/or sequencing as provided in the methods and assays of the present invention. Various methods for the isolation or procurement of nucleic acid may be employed, as any skilled artisan may know and practice. Such methods may include methods employed for the isolation of DNA in various forms and states of purity and may not necessarily involve or require the separation of DNA from all cellular debris, protein, etc. The term isolation as used herein is contemplated to include the preparation of cell or tissue samples whereby DNA may be analyzed, amplified, etc. in situ.

The step (b) may be performed utilizing any method of amplification, including polymerase chain reaction (PCR), ligase chain reaction (Barany, F. (1991) Proc. Natl. Acad. Sci. 88:189-193), rolling circle amplification (Lizardi, P. M. et al (1998) Nature Genetics 19:225-232), strand displacement amplification (Walker, G. T. et al (1992) Proc. Natl. Acad. Sci. 89:392-396) or alternatively any means or method whereby concentration or sequestration of sufficient amounts of the S. mutans and/or S. sanguinis nucleic acid for analysis may be obtained. The primers for use in amplification of S. mutans and/or S. sanguinis nucleic acid may be selected and utilized by the skilled artisan employing the publicly available or herein disclosed bacterial sequences and the sequence of the Sm479F/Sm479R targeted region of S. mutans and/or the Sa475F/Sa475R targeted region of S. sanguinis, including as provided herein in FIG. 4 and FIG. 5, respectively. Particular exemplary primers are provided herein and include oligonucleotide primers having the sequence set out in SEQ ID NOS: 1, 2, 3, 4, 6, 7, 18 and 19. Based on the sequences known and as provided herein, PCR primers are constructed that are complementary to the Sm479F/Sm479R targeted region of S. mutans (SEQ ID NOS: 9-16) and/or the Sa475F/Sa475R targeted region of S. sanguinis (SEQ ID NO: 17), and particularly to a portion thereof.

A primer consists of a consecutive sequence of polynucleotides complementary to any region in the allele encompassing the position which is mutated in the mutant allele. The size of these amplification/PCR primers range anywhere from five bases to hundreds of bases. However, the preferred size of a primer is in the range from 10 to 40 bases, most preferably from 15 to 35 bases. As the size of the primer decreases so does the specificity of the primer for the targeted region. Hence, even though a primer which is less than five bases long will bind to the targeted region, it also has an increased chance of binding to other regions of the template polynucleotide which are not in the targeted region and do not contain the polymorphic/mutated base. Conversely, a larger primer provides for greater specificity, however, it becomes quite cumbersome to make and manipulate a very large fragment. Nevertheless, when necessary, large fragments are employed in the method of the present invention. To amplify the Sm479F/Sm479R targeted region of S. mutans and/or the Sa475F/Sa475R targeted region of S. sanguinis in a sample or subject, primers to one or both sides of the targeted position or internal in the targeted region, are made and used in a PCR amplification reaction, using known methods in the art (e.g. Massachusetts General Hospital & Harvard Medical School, Current Protocols In Molecular Biology, Chapter 15 (Green Publishing Associates and Wiley-Interscience 1991) and as particularly exemplified herein.

The (c) analysis step will utilize skills and methods available to the skilled artisan for determining and distinguishing a sequence and can include: direct sequencing of the amplified or otherwise sequestered product; hybridization utilizing a labeled probe or labeled probe, including as provided herein; direct visualization of the PCR product by gel separation; or by the presence of a non-radioactive dye or fluorescent dye introduced with the primer, (including fluorescence as provided by the molecular beacon technology (Tyagi, S, and Kramer, F. (1996) Nature Biotech 14:303-308; Tyagi, S. et al (1998) Nature Biotech 16:49-53); restriction enzyme analysis wherein restriction enzyme cleavage is characteristic of the bacterial sequence; sequencing by hybridization, etc.

Following amplification, the PCR product may be sequenced, subjected to a second round of amplification, or otherwise analyzed in step (c). The sequence may be determined using any of various methods known in the art, including but not limited to traditional sequencing methodologies and more rapid and high throughput mini-sequencing or pyrosequencing, including but not limited to those exemplified in Cai et al, Sun et al and Ahmadian et al, which references are incorporated herein in their entirety by reference (Cai, H al (2000) Genomics 66(2):135-143; Sun, X et al (2000) Nucleic Acids Res 28(12):E68; Ahmadian, A. et al (2000) Anal Biochem 280(1):103-110). In utilizing certain of these particularly sensitive and efficient sequencing methodologies it may, in fact, not be necessary to perform the (b) amplification step, provided that suitable starting nucleic acid is isolated in (a) for analysis. By utilizing methods which do not require sequencing and whereby the presence of the Sm479F/Sm479R targeted region of S. mutans and/or the Sa475F/Sa475R targeted region of S. sanguinis can be directly determined or inferred, one can rapidly screen samples or DNA isolated from many individuals. A two step or two round amplification approach has been used successfully to examine polymorphisms in the CYP2D6 and in thiopurine methyltransferase (TPMT) genes (Evans et al, Pharmacogenetics, 1: 143_{—}148, 1991.). A nested PCR approach can be utilized. The first round of amplification is used to amplify the gene segment that may contain the sequence of interest. The second round of amplification can utilize one of the common primers as its first primer but importantly makes use of a second primer for either the wild type sequence or for the mutation of interest, and using PCR conditions that produce sequence specific amplification. Alternatively, restriction enzyme digestion of the first or second round PCR product may be used to detect the presence or absence of a mutation, when a restriction site is either gained or lost. The second round of amplification may utilize an allele specific system, for instance, a mismatch directed primer, wherein a base change is specifically introduced by the primer, thereby generating a restriction site at or near the site of the point mutation in a particular allele. Alternatively, an allele specific oligonucleotide, ligase chain reaction, etc. may be utilized so as to generate product only in the presence of a particular base or provide products which are distinguishable by dye, label, size, etc. in each case.

In accordance with the present invention, a diagnostic assay is provided for determining the presence or amount of S. mutans in a sample or subject which comprises

(a) isolating nucleic acid from said sample or subject;
(b) amplifying Sm479F/Sm479R targeted & mutans sequence or a portion thereof using PCR or other amplification technology; and
(c) determining the presence and amount of the Sm479F/Sm479R PCR product sequence, thereby determining the presence or amount of S. mutans in said sample or subject.

In accordance with the present invention, a diagnostic assay is provided for determining the presence or amount of S. mutans in a sample or subject which comprises

(a) isolating nucleic acid from said sample or subject;
(b) amplifying Sm479F/Sm479R targeted S. mutans sequence PCR fragment from said nucleic acid; and
(c) detecting and quantitating the amplified Sm479F/Sm479R targeted S. mutans sequence or portion thereof obtained in step (b), thereby determining the presence or amount of & mutans in said sample or subject.

In accordance with the present invention, a diagnostic assay is provided for determining the presence or amount of S. sanguinis in a sample or subject which comprises

(a) isolating nucleic acid from said sample or subject;
(b) amplifying Sa475F/Sa475R targeted S. sanguinis sequence or a portion thereof using PCR or other amplification technology; and
(c) determining the presence and amount of the S. sanguinis Sa475F/Sa475R targeted PCR product sequence, thereby determining the presence or amount of S. sanguinis in said sample or subject.

In accordance with the present invention, a diagnostic assay is provided for determining the presence or amount of S. sanguinis in a sample or subject which comprises

(a) isolating nucleic acid from said sample or subject;
(b) amplifying Sa475F/Sa475R targeted S. sanguinis sequence PCR fragment from said nucleic acid; and
(c) detecting and quantitating the amplified Sa475F/Sa475R targeted S. sanguinis sequence or portion thereof obtained in step (b), thereby determining the presence or amount of S. sanguinis in said sample or subject.

In accordance with the present invention, a diagnostic assay is provided for simultaneously determining the presence or amount of S. mutans and S. sanguinis in a sample or subject which comprises

(a) isolating nucleic acid from said sample or subject;
(b) amplifying both Sm479F/Sm479R targeted S. mutans sequence PCR fragment and Sa475F/Sa475R targeted S. sanguinis sequence PCR fragment from said nucleic acid; and
(c) detecting and quantitating both of the amplified portion of Sm479F/Sm479R targeted S. mutans sequence and the amplified portion of Sa475F/Sa475R targeted S. sanguinis sequence obtained in step (b), thereby determining the presence or amount of both S. mutans and S. sanguinis in said sample or subject.

In an aspect, the present invention provides a diagnostic assay for simultaneously determining the presence or amount of S. mutans and S. sanguinis in a sample or subject which comprises

(a) isolating nucleic acid from said sample or subject;
(b) amplifying both Sm479F/Sm479R targeted S. mutans sequence PCR fragment and Sa475F/Sa475R targeted S. sanguinis sequence PCR fragment from said nucleic acid using a set of primers, wherein said set of primers contains primer pair X and Y and primer pair A and B; wherein

(i) the X and Y primer pair are complementary to a portion of the Sm479F/Sm479R targeted S. mutans sequence;

(ii) the A and B primer pair are complementary to a portion of the Sa475F/Sa475R targeted S. sanguinis sequence;

(c) amplifying both the sequence in between primers X and Y and the sequence in between primers A and B, thereby obtaining two distinct amplified fragments; and
(d) detecting and quantitating the amplified fragments obtained in step (c), thereby determining the presence or amount of S. mutans and S. sanguinis in said sample or subject.

In a particular example, the present invention extends to a diagnostic assay, wherein primer X has the sequence corresponding to SEQ ID NO: 3, or a fragment thereof which is at least ten bases long, primer Y has the sequence corresponding to SEQ ID NO: 4, or a fragment thereof which is at least ten bases long, primer A has the sequence corresponding to SEQ ID NO: 6, or a fragment thereof which is at least ten bases long, primer Y has the sequence corresponding to SEQ ID NO: 7.

In a further example, the present invention extends to a diagnostic assay, wherein primer X has the sequence corresponding to SEQ ID NO: 1, or a fragment thereof which is at least ten bases long, and primer Y has the sequence corresponding to SEQ ID NO: 2, or a fragment thereof which is at least ten bases long. In a further example, the present invention extends to a diagnostic assay, wherein primer A has the sequence corresponding to SEQ ID NO: 18, or a fragment thereof which is at least ten bases long, and primer B has the sequence corresponding to SEQ ID NO: 19, or a fragment thereof which is at least ten bases long. In a particular example, the present invention extends to a diagnostic assay, wherein primer X has the sequence corresponding to SEQ ID NO: 1, or a fragment thereof which is at least ten bases long, primer Y has the sequence corresponding to SEQ ID NO: 2, or a fragment thereof which is at least ten bases long, primer A has the sequence corresponding to SEQ ID NO: 18, or a fragment thereof which is at least ten bases long, primer Y has the sequence corresponding to SEQ ID NO: 19.

In accordance with the present invention, a diagnostic assay is provided for determining the presence or amount of S. mutans in a sample or subject which comprises

(a) isolating nucleic acid from said sample or subject;
(b) amplifying Sm479F/Sm479R targeted S. mutans sequence or a portion thereof using PCR or other amplification technology in the presence of a fluorogenic probe; and
(c) determining the presence and amount of the Sm479F/Sm479R PCR product sequence, wherein the amount of fluorescence is indicative of the presence and amount of PCR product, thereby determining the presence or amount of S. mutans in said sample or subject.

In a further aspect of the present invention, a diagnostic assay is provided for determining the presence or amount of S. sanguinis in a sample or subject which comprises

(a) isolating nucleic acid from said sample or subject;
(b) amplifying Sa475F/Sa475R targeted S. sanguinis sequence or a portion thereof using PCR or other amplification technology in the presence of a fluorogenic probe; and
(c) determining the presence and amount of the Sa475F/Sa475R targeted PCR product sequence, wherein the amount of fluorescence is indicative of the presence and amount of PCR product, thereby determining the presence or amount of S. sanguinis in said sample or subject.

In a still further aspect, a diagnostic assay is provided for simultaneously determining the presence or amount of S. mutans and S. sanguinis in a sample or subject which comprises

(a) isolating nucleic acid from said sample or subject;
(b) amplifying from said nucleic acid both Sm479F/Sm479R targeted S. mutans sequence PCR fragment in the presence of an S. mutans product specific fluorogenic probe, and Sa475F/Sa475R targeted S. sanguinis sequence PCR fragment in the presence of an S. sanguinis product specific fluorogenic probe, wherein the fluorogenic probes have distinct fluorophores; and
(c) determining the presence and amount of both the Sm479F/Sm479R PCR product sequence and the Sa475F/Sa475R S. sanguinis sequence PCR fragment, wherein the amount of fluorescence of each fluorophore is indicative of the presence and amount of PCR product, thereby determining the presence or amount of both S. mutans and S. sanguinis in said sample or subject.

In one embodiment, the above assays utilize a set of Sm479F/Sm479R targeted S. mutans primers selected from SEQ ID NO: 1 and 2 or SEQ ID NO: 3 and 4 and a fluorogenic probe sequence of SEQ ID NO: 5. In a further embodiment, the above assays utilize a set of Sa475F/Sa475R targeted S. sanguinis primers selected from SEQ ID NO: 6 and 7 or SEQ ID NO: 18 and 19 and a fluorogenic probe sequence of SEQ ID NO: 8.

In a further embodiment of this invention, commercial test kits suitable for use by a medical specialist or dentist may be prepared to determine the presence or amount of S. mutans and/or S. sanguinis in a sample or subject. In accordance with the testing techniques discussed herein, one class of such kits will contain at least the labeled probe or its binding partner, and directions, of course, depending upon the method selected. The kits may also contain peripheral reagents such as buffers, stabilizers, etc.

The present invention thus provides a test kit for determining the presence or amount of S. mutans and/or S. sanguinis in a sample or subject, comprising:

(a) a predetermined amount of a first PCR primer set which amplifies Sm479F/Sm479R targeted S. mutans sequence or a portion thereof;
(b) a predetermined amount of a second PCR primer set which amplifies Sa475F/Sa475R targeted S. sanguinis sequence or a portion thereof;
(c) other reagents, optionally including a fluorogenic probe specific for the amplified Sm479F/Sm479R targeted S. mutans PCR product and a distinct fluorogenic probe specific for the amplified Sa475F/Sa475R targeted S. sanguinis PCR product; and
(d) directions for use of said kit.

In a particular embodiment, the first PCR primer set in a test kit of the present invention has sequences corresponding to SEQ ID NO: 1 and SEQ ID NO: 2, or SEQ ID NO: 3 and SEQ ID NO:4, or fragments thereof which are at least ten bases long.

In a further particular embodiment, the second PCR primer set in a test kit of the present invention has sequences corresponding to SEQ ID NO: 6 and SEQ ID NO: 7, or SEQ ID NO: 18 and SEQ ID NO: 19, or fragments thereof which are at least ten bases long.

In a still further particular embodiment, the S. mutans Sm479F/Sm479R product fluorogenic probe has a sequence corresponding to SEQ ID NO: 5, or a fragment thereof which is at least ten bases long. In another particular embodiment, the S. sanguinis Sa475F/Sa475R product fluorogenic probe has a sequence corresponding to SEQ ID NO: 8, or a fragment thereof which is at least ten bases long.

In a further aspect, the present invention provides oligonucleotide primers or probes suitable for use in the determination of the presence or amount of S. mutans and S. sanguinis in a sample or subject.

The invention provides an isolated oligonucleotide primer having a sequence selected from SEQ ID NO: 1, 2, 3, 4, 6 or 7, or a fragment thereof which is at least ten bases long. The invention provides a composition of a primer pair and probe set comprising the sequences SEQ ID NO: 3, 4 and 5 in combination suitable for amplification and detection of S. mutans in a sample. The invention provides a composition of a primer pair and probe set comprising the sequences SEQ ID NO: 6, 7 and 8 in combination suitable for amplification and detection of S. sanguinis in a sample. In a further embodiment, the invention includes a composition of primer pairs and probes in combination, suitable for simultaneous amplification and detection of S. mutans and S. sanguinis in a sample, comprising the sequences SEQ ID NO: 3, 4, 5, 6, 7 and 8.

In an aspect of the invention, the fluorophore on the fluorogenic probe(s) may be selected from 6-carboxyfluoroscein (FAM), 2′-chloro-7′-phenyl-1,4-dichloro-6-carboxyfluorescein (VIC), tetrachloro-6-carboxyfluorescein, and hexachloro-6-carboxyflorescein. In a further aspect, the fluorogenic probe(s) further comprise a covalently attached quencher. The quencher may be selected from a non-fluorescent quencher, such as a minor groove binder, and a fluorescent quencher, such as 6-caboxytetramethylrhodamine (TAMRA).

Also, antibodies including both polyclonal and monoclonal antibodies, and drugs that modulate the growth or prevalence of S. mutans and/or S. sanguinis may possess certain diagnostic applications and may for example, be utilized for the purpose of detecting and/or measuring conditions such as dental caries, dental disease or the like. For example, the bacteria or their unique sequence regions may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the growth or prevalence of S. mutans and/or S. sanguinis may be discovered or synthesized, and may be used in diagnostic and/or therapeutic protocols.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference.

As used herein, “pg” means picogram, “ng” means nanogram, “ug” or “μg” mean microgram, “mg” means milligram, “ul” or “μl” mean microliter, “ml” means milliliter, “1” means liter.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host. Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage λi, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast α-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered. In selecting an expression control sequence, a variety of factors will normally be considered, including, for example, the relative strength of the system, its controllability, and its compatibility with the particular DNA sequence or gene to be expressed, particularly as regards potential secondary structures.

It is further intended that analogs and sequence variations may be prepared from nucleotide sequences, oligonucleotides, primers and probes within the scope of the present invention. Analogs, such as fragments, may be produced, for example, by digestion or the nucleic acid. Other analogs, such as muteins, can be produced by standard site-directed mutagenesis of the nucleotide sequences. The suitability of these analogs and variants may be tested in the assays and methods of the invention.

As mentioned above, a DNA sequence of use in the invention can be prepared synthetically or by any such other means known or acceptable in the art.

The labels most commonly employed for these studies are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. The sequences, primers and probes can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized.

In accordance with the above, an assay system for screening potential drugs effective to modulate presence or amount of S. mutans and/or S. sanguinis in a sample or subject may be prepared. The bacteria may be introduced into a test system, and the prospective drug may also be introduced into the resulting cell culture, and the culture thereafter examined to observe any changes in the presence or amount of S. mutans and/or S. sanguinis in the sample or subject, due to the addition of the prospective drug or drugs in combination.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

Streptococcus mutans is the major microbial pathogen associated with dental caries in children. The objectives of this study were to design and evaluate species-specific primers for the identification of S. mutans. Validation of the best primer set, Sm479F/R, was performed using 7 S. mutans reference strains, 48 ATCC non-S. mutans strains, 92 S. mutans clinical isolates, mixed DNA samples of S. mutans-S. sobrinus or S. mutans-S. sanguinis, and total cultivable bacterial DNA of saliva samples from 33 18-month-old children. All of the S. mutans samples tested positive, and no PCR products were amplified from members of the other streptococci or non-streptococci strains examined. The lowest detection level for PCR was 10⁻² ng of S. mutans DNA (c. 4.6×10³ copies) in the test samples. The results of our study suggest that the Sm479F/R primer pair is highly specific and sensitive for identification of S. mutans in either purified or mixed DNA samples.

### Introduction

Dental caries is an infectious disease with S. mutans being the major pathogen. Conventionally, studies of S. mutans have relied heavily upon cultivation to identify and characterize S. mutans in the oral cavity. The major limitations of culture methods include a finite threshold of detection of S. mutans in clinical samples; an inconsistent morphology depending on the culture medium used; and its high cost and labor intensiveness. Moreover, cultivation requires viable samples, making its application in field epidemiological studies and high-throughput research impractical.

Because conventional culture methods can limit population-based field studies of S. mutans colonization and its interaction with other bacteria in the oral cavity, a number of DNA-based probes and primers have been developed. Many of the specific probes or primers were targeted to specific genes that are associated with virulence in S. mutans, such as glucosyltransferases (Colby et al., 1995; Yano et al., 2002), fructosyltransferases (Smorawinska & Kuramitsu, 1992), dextranase (Igarashi et al., 1996), glucan-binding protein B (Smith et al., 2003), cell surface protein (Lee & Boran, 2003), the phosphoenolpyruvate-dependent sucrose phosphotransferase system (Macrina et al., 1991; Cvitkovitch et al., 1995), and protein antigen (Okahashi et al., 1989; Okahashi et al., 1993). Several other sets of primers for PCR were designed to amplify specific regions of the 16S rRNA genes of S. mutans (Bentley et al., 1991; Shiroza et al., 1998; Oho et al., 2000; Rupf et al., 2001; Aguilera Galaviz et al., 2002; Becker et al., 2002; Wang et al., 2002; Yano et al., 2002; Yoshida et al., 2003; Arakawa et al., 2004; Hoshino et al., 2004). After an extensive literature review, we found that many primers for PCR assays work well for pure S. mutans cultures. However, there was very little information as to whether the PCR-targeted regions might also be present in other bacterial species found in the same habitat as S. mutans or whether these primers can detect S. mutans in mixed clinical specimens. Indeed, some of these genetic loci may not be unique to S. mutans (Hamada & Slade, 1980; Russell, 1991).

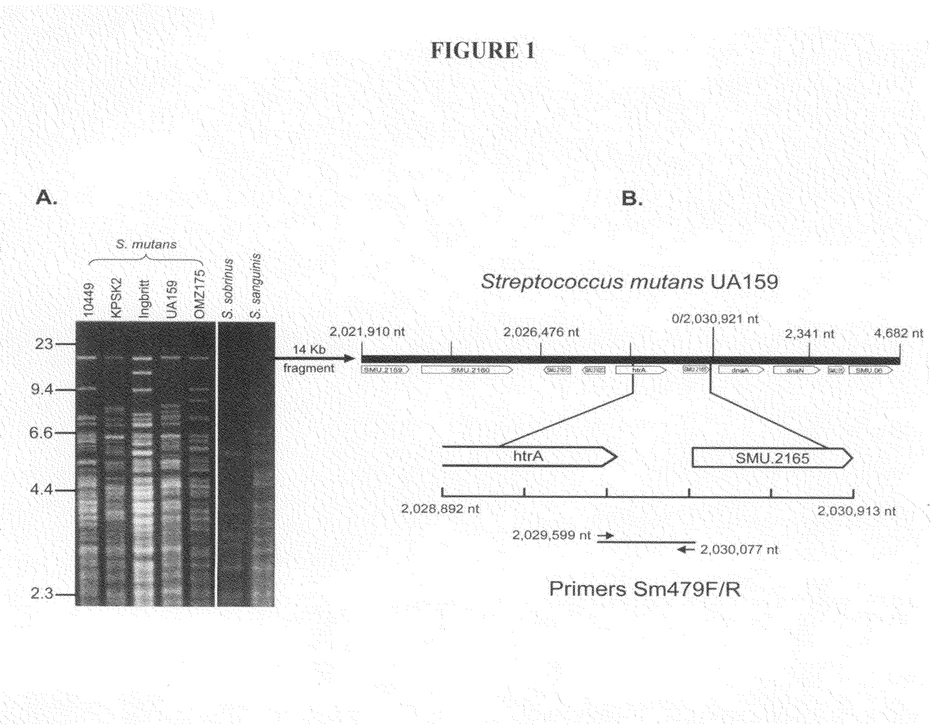

Previously, the maternal influence on mother-to-child transmission of S. mutans was investigated using a chromosomal DNA fingerprinting technique in various populations (Li & Caufield, 1995; Emanuelsson et al., 1998; Li et al., 2000; Li et al., 2001; Caufield et al., 2007). In hundreds of S. mutans chromosomal DNA fingerprints we observed the consistent presence of a 14-kilobase HaeIII restriction fragment as illustrated in FIG. 1A. The main objective of the present study was to identify unique sequence information in this 14-kb fragment for development of S. mutans-specific PCR primers. One such primer pair, Sm479F/Sm479R, was evaluated for its sensitivity and specificity for S. mutans in various mixed bacterial samples. The PCR results were further compared against the findings from conventional culture methods archived in a natural history database (Li et al., 2005a).

### Materials and Methods

This study protocol was approved by the IRB of the University of Alabama at Birmingham on Activities Involving Human Subjects and the IRB of New York University. Written parental consent was obtained for each child in this study.

### Bacterial Samples

Four sets of bacterial samples were included in this study.

(1) Based on their association with dental diseases, a variety of bacterial reference strains (mol % of G+C content ranged from 27% to 71%) were selected (Table 1): 7 S. mutans reference strains, 16 other streptococci reference strains, and 32 non-streptococci gram-positive and gram-negative oral bacteria reference strains. All but 11 of the DNA samples were isolated using a commercial DNA extraction kit (Genomic-tip 100/G, QIAGEN, Valencia, Calif.) followed by an additional phenol-chloroform-isoamyl alcohol extraction. Genomic DNA samples of 11 of the 55 reference strains, indicated with “D” in Table 1, were directly purchased from American Type Culture Collection (ATCC, Manassas, Va.).

(2) For testing the specificity and sensitivity of the primer sets, 92 clinical isolates of S. mutans were randomly selected from our archived S. mutans collection. All were confirmed previously as being S. mutans based on both phenotypic and genotypic profiles (Li & Caufield, 1995; Li et al., 2001; Li et al., 2005a). The genomic DNAs of these isolates were purified using the same DNA extraction kit above.

(3) Mixtures of DNA samples of S. mutans plus S. sobrinus and S. mutans plus S. sanguinis were prepared. A serial dilution (10 ng/μl to 10⁻³ ng/μl) of purified DNA samples of S. mutans (UA159) was added into known concentrations of genomic DNA samples of either S. sobrinus (OMZ65) or S. sanguinis (ATCC10556). The mixed samples were used to determine, by PCR, the lowest detectable concentration of S. mutans DNA in the presence of other oral streptococcus species.

(4) Thirty-three bacterial samples obtained from a MM10-sucrose blood medium (Syed & Loesche, 1973) were also selected for testing the species specificity and the limit of detection of the new primer set for identifying S. mutans in mixed bacterial samples. These saliva samples were previously collected from 33 18-month-old children; 5 of the 33 children (15%) were positive for S. mutans by using a conventional culture assay. The procedures for sample collection, bacterial cultivation, and DNA isolation were published elsewhere (Li et al., 2005a; Li et al., 2005b).

PCR and Real-Time Quantitative PCR Assays (Real-Time qPCR)

The species specificity of each newly developed primer set was evaluated initially against the 7 S. mutans and 48 non-S. mutans reference strains (Table 1), and further validated using the randomly selected purified S. mutans DNA of clinical isolates and the mixed bacterial samples described above. The limit of detection of the primers was evaluated using PCR against a set of 10-fold serially diluted concentrations of UA 159 genomic DNA samples and further validated using the mixed S. mutans DNA samples containing known concentrations of Streptococcus sobrinus or Streptococcus sanguinis DNA.

TABLE 1
List of bacterial samples used in this study

| Bacterial species | Sources and code | Sources of isolation and clinical significance |
|---|---|---|
| Streptococcus mutans strains: | | |
| S. mutans UA159 | ATCC 700610* | caries-active child |
| S. mutans NCTC10449 | ATCC 25175 | human carious dentine |
| S. mutans AF199 | This study | caries-active child |
| S. mutans Ingbritt | B. Krasse^{†} | dental plaque of highly caries-active person |
| S. mutans GS5 | R. J. Gibbons^{‡} | human carious lesions |
| S. mutans LM7 | R. J. Gibbons | caries-active child |
| S. mutans OMZ175 | B. Guggenheim^{§} | human carious lesions |
| Non-Streptococcus mutans strains | | |
| S. agalactiae | ATCC BAA-611D | human clinical specimen |
| S. criceti AHT | B. Krasse | human dental plaque |
| S. cristatus | ATCC 49999 | human oral cavity and throat |
| S. gordonii | ATCC 10558 | patient with bacterial endocarditis |
| S. oralis | ATCC 10557 | patient with bacterial endocarditis |
| S. oralis | ATCC 9811 | human mouth |
| S. parasanguinis | ATCC 15911 | human throat |
| S. pyogenes | ATCC 12344D | human pharyngitis |
| S. ratti | ATCC 19645 | caries lesion in rat |
| S. ratti BHT | T. Shiota^{Δ} | caries lesion in rat |
| S. salivarius | ATCC 7073 | patient with acute articular rheumatism |
| S. sanguinis | ATCC 10556 | patient with bacterial endocarditis |
| S. sobrinus OMZ176 | B. Guggenheim | human carious lesions |
| S. sobrinus OMZ65 | B. Guggenheim | human carious lesions |
| S. sobrinus | ATCC 33478 | human dental plaque |
| S. vestibularis | ATCC 49124 | human oral cavity |
| Gram-positive rods | | |
| Actinomyces naeslundii | ATCC 12104 | human sinus |
| A. odontolyticus | ATCC 17929 | deep carious lesions around teeth |
| A. viscosus | ATCC 15987 | naturally occurring periodontal disease in hamsters |
| A. israelii | ATCC 12102 | human brain abscess |
| A. meyeri | ATCC 35568 | human with purulent pleurisy |
| A. gerencseriae | ATCC 29322 | cervicofacial actinomycosis |
| A. odontolyticus | ATCC 29323 | dental plaque |
| A. georgiae | ATCC 49285 | healthy subgingival plaque |
| A. radingae | ATCC 51856 | human perianal abscess |
| A. bovis | ATCC 13683 | typical case of lumpy jaw in a cow |
| A. bernardiae | ATCC 51728 | human eye infection |
| Bifidobacterium infantis | ATCC 15697D | intestine of infant |
| Lactobacillus casei | ATCC 393 | dairy products (cheese) |
| L. rhamnosus | ATCC 7469 | human infective endocarditis and bacteremia |
| L. salivarius subsp. salivarius | ATCC 11741 | oral cavity |
| L. casei | ATCC 11578 | oral cavity |
| L. fermentum | ATCC 14931 | fermented beets |
| L. paracasei subsp. paracasei | ATCC 25598 | milking machine |
| L. acidophilus | ATCC 4356 | human mouth |
| Gram-negative cocci | | |
| Veillonella parvula | ATCC 10790D | intestinal tract |
| Gram-negative rods | | |
| Actinobacillus | ATCC 43718 | subgingival dental plaque |
| actinomycetemcomitans | | |
| Act. actinomycetemcomitans | ATCC 29522 | mandibular abscess |
| Campylobacter jejuni subsp. jejuni | ATCC 33560D | feces, animal (bovine feces) |
| Escherichia coli | ATCC 10798D | feces from diphtheria convalescent |
| Fusobacterium nucleatum subsp. | ATCC 49256 | human periodontal pocket |
| vincenti | | |
| F. nucleatum subsp. polymorphum | ATCC 10953 | inflamed gingiva, adult male |
| Aggregatibacter | ATCC 700685D | subgingival plaque with juvenile periodontitis |
| actinomycetemcomitans | | |
| Helicobacter pylori | ATCC 43504D | human gastric antrum |
| Prevotella intermedia | ATCC 25611D | empyema |
| Porphyromonas gingivalis | ATCC 33277 | human gingival sulcus |
| Shigella flexneri | ATCC 29903D | pathogen of acute gastroenteritis |
| Tannerella forsythensis | ATCC 43037D | human periodontal pocket |
| Additional bacterial samples | | |
| S. mutans + S. sobrinus | This study | pure culture mixed |
| S. mutans + S. sanguinis | This study | pure culture mixed |
| S. mutans clinical isolates | This study | N = 92; randomly selected from archived bacterial database |
| DNA of total cultivable bacteria in | This study | N = 33; salivary samples of children aged 18 months |
| saliva | | |
| Human DNA samples | | |
| genomic DNA | This study | whole blood cells |
| genomic DNA | This study | buccal mucosa epithelial cells from oral cavity |

*American Type Culture Collection, Manassas, VA, USA. The code with “D” at the end indicates that DNA samples were directly purchased.
^{†}Department of Cariology, Faculty of Odontology, University of Goteborg, Goteborg, Sweden.
^{‡}Forsyth Dental Center, Boston, MA.
^{§}Institute for Oral Biology, Section for Oral Microbiology and General Immunology, University of Zurich, Zurich, Switzerland.
^{Δ}Department of Microbiology, University of Alabama, Birmingham, AL.

PCR assays were performed using a standardized protocol in a thermal cycler (GeneAmp PCR system 9700, Applied Biosystems, Foster City, Calif.). Each reaction mixture (25 μl total volume) contained 1×PCR buffer (10 mM Tris-HCl, 50 mM KCl, pH 8.3), 1.5 μl of 2.5 mM dNTP mixture, 1 mM MgCl₂, 10 pmoles each of forward and reverse primers, 1.5 U of Taq DNA polymerase, and 10 ng of template DNA. The reaction was conducted as follows: 95° C. for 2 min, followed by 40 cycles of 95° C. for 30 s, 60±5° C. for 30 s, and 72° C. for 1 min, then finally 5 min at 72° C. for extension. The PCR amplicons were evaluated in a 1.5% agarose gel in TBE (Tris-borate-EDTA) buffer and stained with ethidium bromide solution (1 μg/ml). The final images of the gels were captured by a digital camera (AlphaImager 3300 System, Alpha Innotech Corp., San Leandro, Calif.).

The species specificity and limit of detection of the primers in identifying S. mutans colonization in 33 18-month-old children was determined using real-time quantitative PCR (real-time qPCR). Briefly, real-time qPCR was performed using an Opticon real-time machine (Monitor-2, MJ Research Inc., Alameda, Calif.) with low-profile 96-well polypropylene microplates. Tenfold serially diluted, known DNA concentrations of S. mutans UA159 were used as an external standard for absolute quantification. Each tube contained 25 μl of reaction mixture, including 1× PCR Master Mix (QuantiTect SYBR Green, Qiagen Inc.), 10 to 100 ng of the mixed bacterial DNA samples obtained from MM10 culture plates and 0.4 μM of each primer. The cycling conditions were 15 min at 95° C. for activation of HotStar Taq DNA polymerase, 45 cycles of 15 s at 94° C. for denaturation, 30 s at 56° C. for annealing and 30 s at 72° C. for extension, followed by a melting curve analysis of the PCR product. All reactions were carried out in duplicate and the final analysis was based on the mean of the two reactions. Furthermore, the PCR products were reconfirmed for correct size by electrophoresis in a 1.5% agarose gel alongside molecular size standards. The real-time qPCR results were compared with the results previously obtained using conventional culture methods.

### DNA Sequencing Analysis

To further confirm the species specificity of the primers, 50% of the PCR products of the S. mutans reference strains, the clinical isolates, and the mixed bacterial DNA samples were randomly selected, purified, and sequenced from both directions (ABI Prism cycle sequencing kit, BigDye Terminator chemistries with AmpliTaq DNA polymerase FS; Perkin-Elmer, Wellesley, Mass.). A sequence similarity search of the nonredundant GenBank database was performed using the standard nucleotide-nucleotide BLAST (National Center for Biotechnology Information, NCBI) search, and sequences were aligned using ClustalW (Chema et al., 2003).

### Statistical Analyses

Analyses were performed using a computer statistics program (SPSS 13.0, SPSS Inc. Chicago, Ill.). The differences in the species specificity and the limit of detection between the different bacterial samples were evaluated using Pearson chi-square and Fisher's Exact Tests. All P values of less than 0.05 were 2-tailed.

### Results

As illustrated in FIG. 1A, a unique 14-kb fragment is present in chromosomal DNA fingerprints of S. mutans isolates after HaeIII restriction enzyme digestion. This fragment was further characterized according to the HaeIII restriction site map of the whole genome sequence of the S. mutans reference strain UA159 (AE014033) using Sequencher Software version 4.1 (Gene Codes Corporation, Ann Arbor, Mich.). The precise length of this unique fragment is 13,693 bp, which encompasses the end of the circular genome (nt 2,021,910-2,030,921) and the contiguous starting region (nt 1-4,682) (FIG. 1B). The protein map of UA159 revealed 100RF within this region, including core housekeeping genes involved with the origin and execution of DNA replication. Because each of the ORF shared some degree of similarity to homologues in other streptococci, we decided to construct primers that spanned from known ORFs to within intergenic spacer regions (ISR). Based on the sequence information, six sets of primers were designed (Table 2) using Primer3 software (Rozen & Skaletsky, 2000) and evaluated against a panel of prototype strains for species specificity.

TABLE 2

Sequences of the candidate primers-designed from
the 13,693-bp HaeIII restriction fragment of S. mutans UA159

| | Oligonucleotide | | Size of |
|---|---|---|---|
| Name | sequence (5′ → 3′) | Base position* (nt) | amplicon (bp) |
| Sm352F | AGC AGA TGG GAA GCT GAA GA | 2,029,470-2,029,489 | 352 |
| Sm352R | GTG GCG CTT AGA ACC CAT TA | 2,029,821-2,029,802 | |
| Sm377F | GAG AAC CAT AAT CCC AGT CTT ATT TT | 2,029,701-2,029,726 | 377 |
| Sm377R | GCC CCT TCA CAG TTG GTT AG | 2,030,077-2,030,058 | |
| Sm436F | CCT TGA CCA ACG GAC CAT AG | 2,026,317-2,026,336 | 436 |
| Sm436R | CTT CTT TGA TCC CAG CAG GA | 2,026,752-2,026,733 | |
| Sm476F | GAC CCG CTC TTG GTA TTT CA | 2,029,346-2,029,365 | 476 |
| Sm476R | GTG GCG CTT AGA ACC CAT TA | 2,029,821-2,029,802 | |
| Sm479F | TCG CGA AAA AGA TAA ACA AAC A | 2,029,599-2,029,621 | 479 |
| Sm479R | GCC CCT TCA CAG TTG GTT AG | 2,030,077-2,030,058 | |
| Sm521F | ACG GCG TAA TCA AAA ACC AG | 2,027,617-2,027,636 | 521 |
| Sm521R | CCC GAT GAG AAA ATT CCA AA | 2,028,137-2,028,118 | |

*Base positions of primers are from nucleotide sequence [GenBank accession no, NC_004350] of genomic DNA derived from Streptococcus mutans UA159.

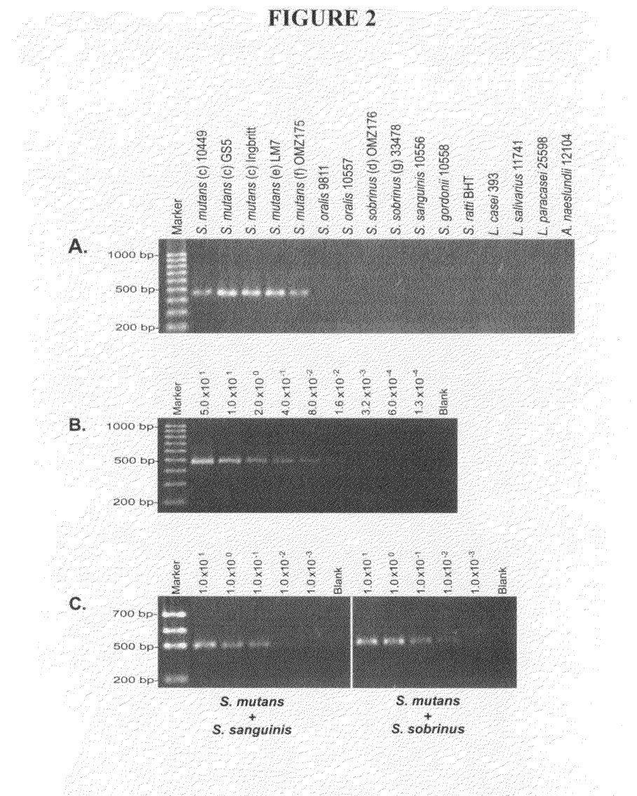

After systematically testing each of the six primer sets, we found that Sm479F: 5′-TCGCGAAAAAGATAAACAAACA-3′ (SEQ ID NO:1) and Sm479R: 5′-GCCCCTTCACAGTTGGTTAG-3′ (SEQ ID NO:2) were highly specific for identification of S. mutans in either purified or mixed DNA samples. The results showed that all of the S. mutans reference strains and S. mutans clinical isolates were PCR-positive. No PCR products were detected in other Streptococcus species, including S. sobrinus, S. criceti, and S. ratti, S. salivarius, S. vestibularis, S. sanguinis, S. parasanguinis, S. gordonii, S. oralis, and S. cristatus or in the other oral bacterial strains (FIG. 2A). In contrast to the Sm479F/R primer pair, the other five sets of primers showed positives among the other streptococci or non-streptococci strains tested. In the serially diluted S. mutans DNA samples, the lowest detectable concentration of the Sm479F/R primers was 0.01 ng/μl (4.6×10³ copies) (FIG. 2B). A similar limit level of detection was also obtained in the S. mutans-S. sanguinis or & mutans-S. sobrinus mixed DNA samples (FIG. 2C).

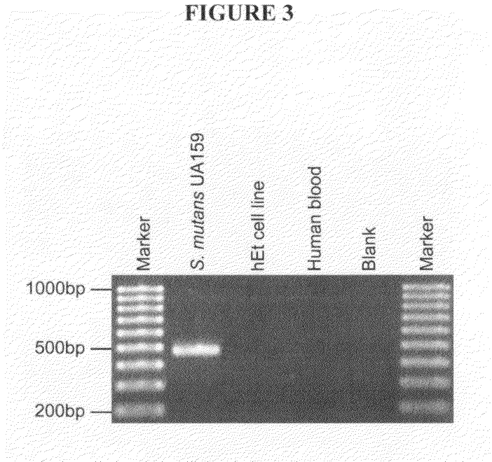

To exclude the possibility that Sm479F/R might encounter unexpected cross-reactivity in reactions applied to mixed clinical samples containing bacterial and human DNA, PCR assays were performed against 10 ng of human genomic DNA sample (isolated from 1 mL blood sample) and 10 ng of hEt cell line DNA sample (derived from human buccal mucosa epithelial cells). Both human DNA samples showed negative PCR results (FIG. 3).

Sequencing analysis revealed that the Sm479F/Sm479R primers target an amplicon of 479-bp (nt 2029599 to 2030077 of AE014133), with a 5′ within the htrA gene and the 3′ within the ISR but outside the putative spoJ gene (SMU.2165; FIG. 1B). Homologues of both genes are widely distributed among Gram-positive bacteria. A search of the nonredundant GenBank database for sequences similar to the 479-bp PCR amplicon yielded only a single hit, which was within the S. mutans UA159 complete genome and showed 100% identity. The results of a multiple sequence alignment revealed that the products amplified by the Sm479F/ Sm479R primers were 98% to 100% identical among the S. mutans serotype c strains UA159, ATCC25175, Ingbritt, and GS5; the serotype e strain LM7; the serotype f strain OMZ175; and randomly selected S. mutans clinical isolates (FIG. 4, SEQ ID NOS: 9-16). The nucleotide sequences targeted by the Sm479F/Sm479R in each serotype of S. mutans have been deposited in GenBank under accession numbers EF533872, EF533873, EF533874, EF533875, EF533876, EF533877, EF533878, and EF533879.

Furthermore, S. mutans DNA was detected by quantitative real-time PCR in 14 of the 33 mixed bacterial samples (42.4%), whereas in the results from culture, 5 of the 33 children (15.2%) had detectable S. mutans in their saliva. All of the samples that tested positive for S. mutans by culturing also tested positive by real-time qPCR (100% agreement). A homogeneous melting peak at 78° C. indicated that the amplified target DNA products were specific for S. mutans. Quantitative real-time PCR with the Sm479F/R primers significantly improved the sensitivity of detecting S. mutans in the clinical samples (42.4% vs. 15.2%; P=0.008; Fisher's Exact Test).

### Discussion

The results of this study demonstrate that the Sm479F/Sm479R primer set is highly sensitive and species-specific for PCR-based detection and evaluation of S. mutans colonization in the oral cavity. The species specificity of the primers was first tested in different types of pure S. mutans DNA samples, and then systematically evaluated and validated in mixed bacterial samples, including S. mutans-S. sobrinus, S. mutans-S. sanguinis, and mixtures of total bacterial colonies from MM10-medium. The reasons for using bacterial samples from MM10-medium were threefold: (1) Culture methods have served as the “gold standard” for bacterial detection for decades. (2) Our previous studies of the colonization of S. mutans, S. sobrinus, and S. sanguinis were all based on conventional culture methods, including the use of MM10-medium. We had full access to a well-archived bacteriological database to conduct various validation experiments, including testing the newly designed S. mutans-specific primers. (3) We utilized the same bacterial samples obtained from the same individuals for the validation tests to minimize, by design, potential experimental bias. We acknowledge that testing of whole saliva samples from the same individuals would yield additional information, but longitudinal samples were not available for this study. Overall, our data demonstrate consistent results among the different sets of bacterial samples; interestingly, the primers can be used to identify not only S. mutans serotype c strains, but also serotype e and f strains. Furthermore, the species specificity was confirmed by DNA sequence analysis. These findings suggest that this S. mutans species-specific primer set is reliable and can be applied to evaluating S. mutans colonization for clinical studies.

The S. mutans-specific primer set was based on the discovery of a unique 14-kb HaeIII restriction fragment of UA 159, though the significance of this fragment present in S. mutans is not well understood. Since the fragment consists of the end of the circular genome with a number of unknown genes and intergenic space regions and a conserved 4-kb segment after the origin, the 14-kb fragment became the starting point for finding a unique signature DNA sequence from S. mutans. In this study we found that the Sm479F/Sm479R primer set targeted region is directly associated with HtrA and genetic competence in S. mutans of UA159 as demonstrated by Ahn, et al. (Ahn et al., 2005). HtrA homologues have been identified in many gram-positive bacteria including streptococci. Most evidence suggests that HtrA acts as a housekeeping protease to degrade unfolded proteins during heat shock (Pallen & Wren, 1997). Biswas and coworkers (Biswas & Biswas, 2005) found that the HtrA protease is associated with the ability to survive under different stress conditions and is essential for stress tolerance, such as high or low temperature and under acidic conditions, in S. mutans. Other studies suggest that htrA may also be involved in the biogenesis of extracellular proteins, biofilm formation, and genetic transformation (Diaz-Torres & Russell, 2001; Ahn et al., 2005; Biswas & Biswas, 2005).

In addition to the htrA gene, the Sm479F/Sm479R primers target an intergenic locus of unknown function that is unique to S. mutans species. PCR amplification of the 16S-23S rRNA intergenic spacer region (IGSR) showed to be a useful tool for bacterial species-specific typing because of the considerable variability in size and sequence among organisms (Bourque et al., 1995; Leys et al., 1999; Kwon et al., 2005; Grattard et al., 2006; Valcheva et al., 2007). We implied the similar assumption, constructed the Sm479F/R to target one of the major IGSR of the 14-kb fragment for species selectivity, and observed a high specificity of the PCR amplification in this study. The particular combination of the 479-bp amplicon, which includes a potential virulence locus (htrA) and a S. mutans species-specific locus (ISR), may offer a new unique biomarker for PCR-based S. mutans identification and S. mutans DNA quantification.

As the conventional culture method is considered to be the “gold standard” for detecting S. mutans colonization, we compared our real-time qPCR results with data previously obtained by culture methods. One of our significant findings is that the real-time qPCR with the Sm479F/R primers significantly increased the sensitivity of detecting S. mutans in clinical samples by nearly threefold. Previously, both Loesche's group and our own study reported that the average detection levels of S. mutans in saliva range from 104 to 106 colony forming units (CFUs) per milliliter using conventional culture methods (Syed & Loesche, 1973; Li et al., 2005a). Oho et al. showed that a PCR method could detect S. mutans in saliva with a detection threshold of >10⁴ CFUs (Oho et al., 2000). In this study, we observed that 64.3% of the children who were S. mutans negative by the culture method were S. mutans positive by real-time qPCR. As little as 10⁻² nanograms of S. mutans DNA, approximately 4.6×10³ of copies of S. mutans, in the saliva that might not be able to produce cultures under standard laboratory conditions, could be detected by PCR. As population-based caries studies begin to move away from culture methods towards recently developed DNA-based molecular methods, it is critical to develop S. mutans-specific primers that can accurately identify and quantify S. mutans in clinical samples. Our findings suggest that the Sm479F/Sm479R primer set has those abilities and may be used for conducting high-throughput epidemiological studies of S. mutans infection and for a better understanding of the microbial role of S. mutans associated with dental caries.

### REFERENCES

Aguilera Galaviz L A, Aceves Medina Mdel C & Estrada Garcia I C (2002) Detection of potentially cariogenic strains of Streptococcus mutans using the polymerase chain reaction. J Clin Pediatr Dent 27: 47-51.
Ahn S J, Lemos J A & Burne R A (2005) Role of HtrA in growth and competence of Streptococcus mutans UA159. J Bacteriol 187: 3028-3038.
Arakawa H, Karasawa K, Igarashi T, Suzuki S, Goto N & Maeda M (2004) Detection of cariogenic bacteria genes by a combination of allele-specific polymerase chain reactions and a novel bioluminescent pyrophosphate assay. Anal Biochem 333: 296-302.
Becker M R, Paster B J, Leys E J, Moeschberger M L, Kenyon S G, Galvin J L, Boches S K, Dewhirst F E & Griffen A L (2002) Molecular analysis of bacterial species associated with childhood caries. J Clin Microbiol 40: 1001-1009.
Bentley R W, Leigh J A & Collins M D (1991) Intrageneric structure of Streptococcus based on comparative analysis of small-subunit rRNA sequences. Int J Syst Bacteriol 41: 487-494.
Biswas S & Biswas I (2005) Role of HtrA in surface protein expression and biofilm formation by Streptococcus mutans. Infect Immun 73: 6923-6934.
Bourque S N, Valero J R, Lavoie M C & Levesque R C (1995) Comparative analysis of the 16S to 23S ribosomal intergenic spacer sequences of Bacillus thuringiensis strains and subspecies and of closely related species. Appl Environ Microbiol 61: 1623-1626.
Caufield P W, Saxena D, Fitch D & Li Y (2007) Population structure of plasmid-containing strains of Streptococcus mutans, a member of the human indigenous biota. J Bacteriol 189: 1238-1243.
Chema R, Sugawara H, Koike T, Lopez R, Gibson T J, Higgins D G & Thompson J D (2003) Multiple sequence alignment with the Clustal series of programs. Nucleic Acids Res 31: 3497-3500.
Colby S M, Harrington D J & Russell R R (1995) Identification and genetic characterisation of melibiose-negative isolates of Streptococcus mutans. Caries Res 29: 407-412.
Cvitkovitch D G, Boyd D A, Thevenot T & Hamilton I R (1995) Glucose transport by a mutant of Streptococcus mutans unable to accumulate sugars via the phosphoenolpyruvate phosphotransferase system. J Bacteriol 177: 2251-2258.
Diaz-Torres M L & Russell R R (2001) HtrA protease and processing of extracellular proteins of Streptococcus mutans. FEMS Microbiol Lett 204: 23-28.
Emanuelsson I R, Li Y & Bratthall D (1998) Genotyping shows different strains of mutans streptococci between father and child and within parental pairs in Swedish families. Oral Microbiol Immunol 13: 271-277.
Grattard F, Ginevra C, Riffard S, Ros A, Jarraud S, Etienne J & Pozzetto B (2006) Analysis of the genetic diversity of Legionella by sequencing the 23S-5S ribosomal intergenic spacer region: from phylogeny to direct identification of isolates at the species level from clinical specimens. Microbes Infect 8: 73-83.
Hamada S & Slade H D (1980) Biology, immunology, and cariogenicity of Streptococcus mutans. Microbiol Rev 44: 331-384.
Hoshino T, Kawaguchi M, Shimizu N, Hoshino N, Ooshima T & Fujiwara T (2004) PCR detection and identification of oral streptococci in saliva samples using gtf genes. Diagn Microbiol Infect Dis 48: 195-199.
Igarashi T, Yamamoto A & Goto N (1996) Rapid identification of mutans streptococcal species. Microbiol. Immunol 40: 867-871.
Kwon H S, Yang E H, Lee S H, Yeon S W, Kang B H & Kim T Y (2005) Rapid identification of potentially probiotic Bifidobacterium species by multiplex PCR using species-specific primers based on the region extending from 16S rRNA through 23S rRNA. FEMS Microbiol Lett 250: 55-62.
Lee S F & Boran T L (2003) Roles of sortase in surface expression of the major protein adhesin P1, saliva-induced aggregation and adherence, and cariogenicity of Streptococcus mutans. Infect Immun 71: 676-681.
Leys E J, Smith J H, Lyons S R & Griffen A L (1999) Identification of Porphyromonas gingivalis strains by heteroduplex analysis and detection of multiple strains. J Clin Microbiol 37: 3906-3911.
Li Y, Caufield P, Emanuelsson I & Thornqvist E (2001) Differentiation of Streptococcus mutans and Streptococcus sobrinus via genotypic and phenotypic profiles from three different populations. Oral Microbiol Immunol 16: 16-23.
Li Y & Caufield P W (1995) The fidelity of initial acquisition of mutans streptococci by infants from their mothers. J Dent Res 74: 681-685.
Li Y, Caufield P W, Dasanayake A P, Wiener H W & Vermund S H (2005a) Mode of delivery and other maternal factors influence the acquisition of Streptococcus mutans in infants. J Dent Res 84: 806-811.
Li Y, Ku C Y, Xu J, Saxena D & Caufield P W (2005b) Survey of oral microbial diversity using PCR-based denaturing gradient gel electrophoresis. J Dent Res 84: 559-564.
Li Y, Wang W & Caufield P W (2000) The fidelity of mutans streptococci transmission and caries status correlate with breast-feeding experience among Chinese families. Caries Res 34: 123-132.
Macrina F L, Jones K R, Alpert C A, Chassy B M & Michalek S M (1991) Repeated DNA sequence involved in mutations affecting transport of sucrose into Streptococcus mutans V403 via the phosphoenolpyruvate phosphotransferase system. Infect Immun 59: 1535-1543.

Oho T, Yamashita Y, Shimazaki Y, Kushiyama M & Koga T (2000) Simple and rapid detection of Streptococcus mutans and Streptococcus sobrinus in human saliva by polymerase chain reaction. Oral Microbiol Immunol 15: 258-262.
Okahashi N, Sasakawa C, Yoshikawa M, Hamada S & Koga T (1989) Cloning of a surface protein antigen gene from serotype c Streptococcus mutans. Mol Microbiol 3: 221-228.
Okahashi N, Takahashi I, Nakai M, Senpuku H, Nisizawa T & Koga T (1993) Identification of antigenic epitopes in an alanine-rich repeating region of a surface protein antigen of Streptococcus mutants. Infect Immun 61: 1301-1306.
Pallen M J & Wren B W (1997) The HtrA family of serine proteases. Mol Microbiol 26: 209-221.
Rozen S & Skaletsky H (2000) Primer3 on the WWW for general users and for biologist programmers. Methods Mol Biol 132: 365-386.
Rupf S, Merte K, Eschrich K, Stosser L & Kneist S (2001) Peroxidase reaction as a parameter for discrimination of Streptococcus mutans and Streptococcus sobrinus. Caries Res 35: 258-264.
Russell R R B (1991) Genetic analysis and genetic probes for oral bacteria. Aspects of oral molecular biology (Ferquson B D, ed). Vol. 8. pp. 57-75. Karger, Basel.
Shiroza T, Shinozaki N, Watanabe T, Ikemi T, Fukushima K & Abiko Y (1998) Rapid isolation of chromosomal DNA from oral streptococci and polymerase chain reaction-oriented restriction fragment-length polymorphism analysis for genetic heterogeneity. Oral Microbiol Immunol 13: 11-16.
Smith D J, King W F, Barnes L A, Peacock Z & Taubman M A (2003) Immunogenicity and protective immunity induced by synthetic peptides associated with putative immunodominant regions of Streptococcus mutans glucan-binding protein B. Infect Immun 71: 1179-1184.
Smorawinska M & Kuramitsu H K (1992) DNA probes for detection of cariogenic Streptococcus mutans. Oral Microbiol Immunol 7: 177-181.
Syed S A & Loesche W J (1973) Efficiency of various growth media in recovering oral bacterial flora from human dental plaque. Appl Microbiol 26: 459-465.
Valcheva R, Kabadjova P, Rachman C, Ivanova I, Onno B, Prevost H & Dousset X (2007) A rapid PCR procedure for the specific identification of Lactobacillus sanfranciscensis, based on the 16S-23S intergenic spacer regions. J Appl Microbiol 102: 290-302.
Wang J, Li C, Xiao B & Liu J (2002) Detection of cariogenic Streptococcus mutans by quantitative polymerase chain reaction. Zhonghua Kou Qiang Yi Xue Za Zhi. 37: 281-283.
Yano A, Kaneko N, Ida H, Yamaguchi T & Hanada N (2002) Real-time PCR for quantification of Streptococcus mutans. FEMS Microbiol Lett 217: 23-30.
Yoshida A, Suzuki N, Nakano Y, Kawada M, Oho T & Koga T (2003) Development of a 5′ nuclease-based real-time PCR assay for quantitative detection of cariogenic dental pathogens Streptococcus mutans and Streptococcus sobrinus. J Clin Microbiol 41: 4438-4441.

### Example 2

### Duplex Real-Time QPCR Analysis of S. Mutans and S. Sanguinis Colonizations in Clinical Samples

Streptococcus mutans is the major microbial pathogens associated with dental caries. Streptococcus sanguinis (formerly S. sanguis), is also a key member of the indigenous oral biota colonizing dental plaque. S. sanguinis is one of the most prevalent members of the oral streptococci, especially on tooth surfaces with no caries present. S. sanguinis may serve a protective or antagonistic role against the cariogenic bacterium S. mutans (Caufield, P. W. et al. (2000) Infect. Immun. 68:4018-4023; Loesche, W. J. and Syed, S. A. (1973) Caries Res. 7:201-216; Loesche, W. J. et al (1973) Arch. Oral Biol. 18:571-575). Based on conventional culture methods, it has been suggested that S. mutansis/S. sanguinis ratio may serve as a caries risk indicator. S. sanguinis may also cause or be associated with bacterial endocarditis and septicemia (Douglas, C. W. et al. (1993) J. Med. Microbiol. 39:179-182; Herzberg, M. C. et al. (1990) Infect. Immun. 58:515-522). To the extent that certain environmental or biological factors may trigger a disruption in the balance of oral bacterial species, leading to microbial diseases, the balance and interaction between S. sanguinis and S. mutans has been studied (Kreth, J. et al (2005) J Bact 187(21):7193-7203). Because relative colonization of S. sanguinis and S. mutans may naturally influence the development and progression of dental caries or dental disease, and the modulation of bacterial colonization or growth may be used to clinically influence the development and progression of dental caries or dental disease, a rapid, reproducible and quantitative assay and test for these bacterial species in a sample or a subject is of significant benefit, both as a tool in development of therapies and for analysis and active use in monitoring, prophylaxis and therapy.

Various references have demonstrated that the proportion and levels of S. mutans in carious tooth surfaces were significantly higher than in caries-free tooth surfaces, while the proportion and levels of S. sanguinis were higher in caries-free tooth surfaces (Loesche W J, et al. (1975) Infect Immun 11(6):1252-1260; Loesche W J and Straffon L H (1979) Infect Immun 26(2):498-507; Marchant S, et al. (2001) Caries Res 35(6):397-406; Nyvad B, and Kilian M (1990) Caries Res 24(4):267-272). S. mutans/S. sanguinis ratio calculation and analysis has been reported and the S. mutans/S. sanguinis ratio was found to be significantly higher in the carious tooth surfaces than in the other tooth surfaces (Loesche W J and Straffon L H (1979) Infect Immun 26(2):498-507). Also, the S. mutans/S. sanguinis ratio changes over time as a function of caries status of teeth, confirming the importance and relevance of S. mutans in the carious tooth surfaces of high caries-active individuals (Loesche W J and Straffon L H (1979) Infect Immun 26(2):498-507).

We have developed and validated species specific probes for duplex PCR for identifying S. mutans (Sm479F/Sm479R) and S. sanguinis (Sa475F/Sa475R, Formerly SSA-2) and PCR-based assays for simultaneous detection of the two species in the mixed bacterial samples. Therefore, the S. mutans/S. sanguinis ratios can be readily obtained. The S. mutans/S. sanguinis ratio or the relative amounts of S. mutans and S. sanguinis bacteria is indicative of the caries status and risk in an individual. The S. mutans probe is directed to a portion of the 479 bp species specific region of S. mutans (Sm479F/Sm479R targeted sequence region) described and detailed in Example 1 and shown in FIG. 4. The particular primers and probe for S. mutans used in this real-time quantitative PCR duplex assay are depicted in Table 4.

The S. sanguinis probe is directed to a portion of the 475 bp SSA-2 region of S. sanguinis (the SSA-2 targeted sequence region, Sa475F/Sa475R, SEQ ID NO: 17). Region SSA-2 (Sa475F/Sa475R), containing only intergenic region sequences between two housekeeping genes uncC (protein-translating AJPase) and murA (UDP-N-acetylglucosamine enolpyruvyl transferase), has been previously identified as specific for S. sanguinis and used as a PCR-generated species-specific DNA probe (Li, Y et al (2003) J Clin Microbiol 41(8): 3481-3486). The sequence of the 475 bp Sa475F/Sa475R(SSA-2) region of S. sanguinis is provided in FIG. 5. The sequence of this Sa475F/Sa475R(SSA-2) intergenic region of S. sanguinis is also publicly available in the GenBank database under accession number AY277586. The particular primers and probe for S. sanguinis used in this real-time quantitative PCR duplex assay are shown in Table 5.

The reference DNA used for standard curves were purified genomic DNA from UA159 for S. mutans and ATCC10556 for S. sanguinis, respectively. The PCR products were read out and quantitatively analyzed using product probes with two different reporter dyes, FAM reporter dye (6-Carboxyfluorescein) for S. mutans and VIC reporter dye (2′-chloro-7′-phenyl-1,4-dichloro-6-carboxyfluorescein) for S. sanguinis. The probes were purchased from Applied Biosystems (Foster, Calif.).

TABLE 4

S. mutans Species-Specific Primers
and Probe for Duplex Real-Time qPCR

| | | Location | Location in |
|---|---|---|---|
| | | in Sm479 | UA159 genome |
| Name | Sequence (5′-3′) | amplicon | (gi:24378526) |
| expsm479-F | AAA TAG ATT AAG ACG TAG AAA AGT | 180-214 | 2029778-2029812 |
| | TAA TGG GTT CT | | |
| | (SEQ ID NO: 3) | | |
| expsm479-R | GTTGTGATGTTATGGAGGACGAGAT | 326-302 | 2029924-2029900 |
| | (SEQ ID NO: 4) | | |
| Probe: | FAM-AAG CGC CAC ATT AAC CA-MGB | 215-231 | 2029813-2029829 |
| expsm479-M1 | (SEQ ID NO: 5) | | |

FAM reporter dye, 6-Carboxyfluorescein;
MGB non-fluorescent quencher, minor groove binder;
The probe targeted final fragment size is 147 bp.

TABLE 5

S. sanguinis Species-Specific Primers
and Probe for Duplex Real-Time qPCR

| | | Location | Location in |
|---|---|---|---|
| | | in Ssa2 | SK36 genome |
| Name | Sequence (5′-3′) | amplicon | (gi:125496804) |
| expsa475-F | CGGTTGTTGAGTGGGAGAGATTTT | 244-267 | 773257-773280 |
| | (SEQ ID NO: 6) | | |
| expsa475-R | CCAAGGCAATGCTAAACAAGAGAAT | 333-309 | 773346-773322 |
| | (SEQ ID NO: 7) | | |
| Probe: | VIC-ATG CGA TGA AAA TTC-MGB | 286-300 | 773299-773313 |
| expsa475-M2 | (SEQ ID NO: 8) | | |

VIC reporter dye, 2′-chloro-7′-phenyl-1,4-dichloro-6-carboxyfluorescein;
MGB non-fluorescent quencher, minor groove binder;
The probe targted final fragment size is 90bp

Duplex Real-Time qPCR.

The duplex real-time quantitative PCR assay was performed by the Opticon Monitor 2 (MJ Research Inc., Alameda, Calif.) using low profile 96-well polypropylene microplates (MJ Research Inc). Serial dilution of UA159 or ATCC10556 genomic DNA was used as an external DNA standard for absolute quantification of either S. mutans or S. sanguinis amounts in clinical samples.

For an optimization of primer concentrations for the duplex real-time qPCR, probes were tested at concentrations between 0.05 and 0.25 μM. Annealing temperatures were tested at 60° C. The final concentration and preparation of 20× primer-probe mixes are listed in Table 6 for easy and reproducible handling, and stored at −20° C. QuantiTech Multiplex PCR NoRox kit (Qiagen, Foster City, Calif.) was used for all amplification reactions. Duplex real-time qPCR amplification mix and conditions are listed in Tables 7 and 8.

TABLE 6
Preparing 20x Primer-Probe Mix from 100 μM Stock
Solution for Duplex real-time qPCR

| | | | |
|---|---|---|---|
| Component | Name | Volume | Concentration |

For analysis of S. mutans

| | | | |
|---|---|---|---|
| Forward primer | expsm479-F | 90 μl | 18 μM |
| Reverse primer | expsm479-R | 90 μl | 18 μM |
| Probe-FAM | expsm479-M1 | 25 μl | 5 μM |
| RNase-free H₂O | | 295 μl | |
| Total volume | | 500 μl | |

For analysis of S. sanguinis

| | | | |
|---|---|---|---|
| Forward primer | expsm475-F | 90 μl | 18 μM |
| Reverse primer | expsa475-R | 90 μl | 18 μM |
| Probe-VIC | expsa475-M1 | 25 μl | 5 μM |
| RNase-free H₂O | | 295 μl | |
| Total volume | | 500 μl | |

TABLE 7
Preparing Reaction Mix for Duplex real-time qPCR for
analysis of S. mutans and S. sanguinis

| Component | Volume | Final concentration |
|---|---|---|
| Reaction mix | | |
| 2 X QuantiTect Multiplex | 12.5 μl | 1 x |
| PCR | | |
| NoRox Master Mix | | |
| 20x Primer-Probe Mix (FAM) | 1.25 μl | primer 0.9 μM/probe 0.25 μM |
| 20x Primer-Probe Mix (VIC) | 1.25 μl | primer 0.9 μM/probe 0.25 μM |
| 1 U/μl Uracil-N-glycosylase | 0.25 μl | 0.25 units/reaction |
| (UNG) | | |
| Template DNA | Variable | |
| RNase-free H₂O | Variable | |
| Total volume per reaction | 25 μl | |

TABLE 8
Cycling Conditions for Duplex real-time qPCR for analysis
of S. mutans and S. sanguinis

| Step | Time | Temperature |
|---|---|---|
| UNG incubation (eliminate contamination) | 2 min | 50° C. |
| PCR Initial activation | 15 min | 95° C. |
| 2-step cycling: | | |
| Denaturation | 15 sec | 95° C. |
| Annealing/extension | 1 min | 60° C. |
| Plate reading | | |
| Number of cycles | 40 | |

### Data Analysis

Real-time qPCR output data were analyzed using Opticon Monitor 2 software (MJ Research Inc). Each of the reporter dyes used (FAM and VIC) was adjusted according to the recommendations provided by the manufacturer.

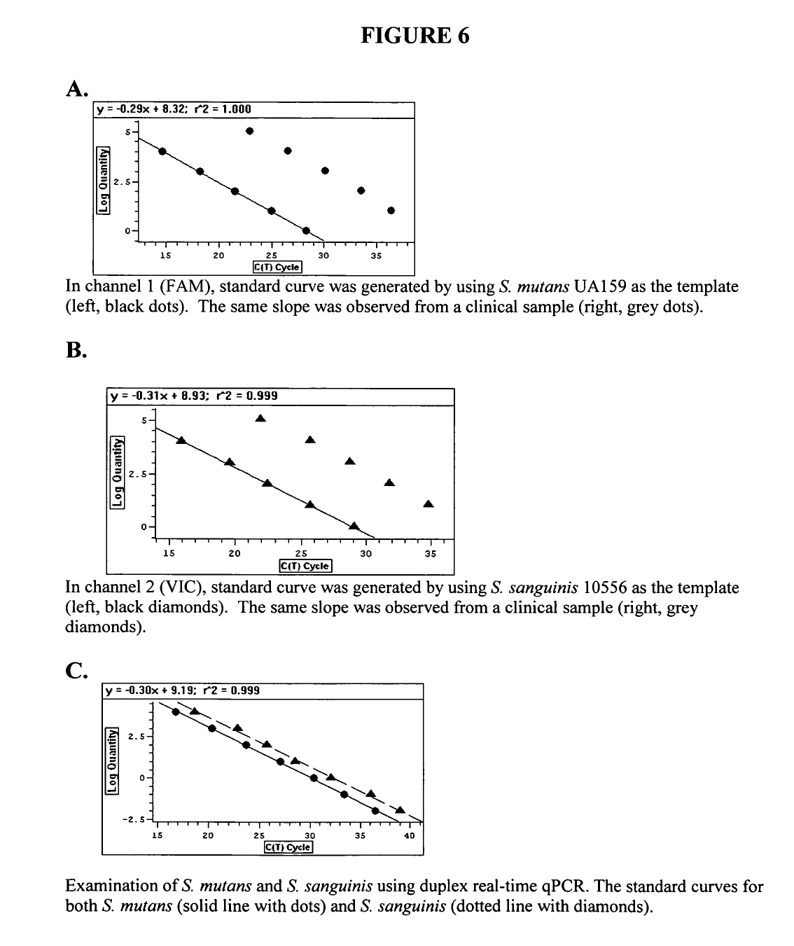

The specificity and minimum detection levels were evaluated by using the same set of known bacterial samples and the same set of clinical samples listed in Table 1 of Example 1. The results showed positive results present only for S. mutans and S. sanguinis strains. Furthermore, the specificity and the minimum detection levels of the duplex qPCR products were confirmed for correct size by electrophoresis in a 1.5% agarose gel. For S. mutans assay, similar C(t) values with “No Background” group were obtained when non-target DNA (either S. sanguinis or S. sobrinus) was added in complex background. In complementary background, similar results with “No Background” were obtained (y=−0.29X+8.83; r²=0.999) (FIG. 6A).

For S. sanguinis assay, similar C(t) values with “No Background” group were obtained when non-target DNA (either S. mutans or S. sobrinus) was added in complex background. In complementary background, similar results with “No Background” were obtained (y=−0.31X+9.12; r²=0.999) (FIG. 6B).

Combined examination of both S. mutans and S. sanguinis is shown in FIG. 6C, with standard curves for both S. mutans and S. sanguinis depicted.

### CONCLUSION

Duplex real-time qPCR with specifically designed primer-probe sets is reliable for analysis of S. mutans and S. sanguinis in both purified and clinical mixed bacterial samples.

### Example 3

### Real-Time (PCR Assay for Detection and Enumeration of S. mutans and S. Sanguinis in Children

Previously and above in Example 1 we reported two sets of primers, Sm479F/Sm479R and Sa475F/Sa475R can be used to detect S. mutans and S. sanguinis by conventional PCR in clinical samples. The purpose of this study is to perform real-time quantitative PCR by applying these S. mutans-specific or S. sanguinis-specific primers in saliva samples of very young children. A total of 584 bacterial genomic DNA samples from 109 children aged from birth to 36 months old were included in this study. Real-time quantitative PCR amplification, data acquisition, and analysis were carried out using the DNA Engine Opticon 2 System (MJ research Inc., Alameda, Calif.). S. mutans UA159 and S. sanguinis ATCC10556 genomic DNA samples were used as the references strain and for the establishment of standard curves for testing clinical samples. Findings of this study show an overall concordance between the PCR assay and conventional cultivation assay was 91.8% and 75.4% for S. mutans and S. sanguinis, respectively. The percentage of children was positive with S. mutans and S. sanguinis was correlated with the children's age, ranging from 3.6% at birth to 36.8% at 36 months of age for S. mutans; 7.3% at birth to 68.4% at 36 months of age for S. sanguinis. PCR assay demonstrated high specificity and significantly improved the positive detection rate for either S. mutans (p<0.05) or S. sanguinis (p<0.05) compared with the culture methods.

Streptococcus sanguinis and Streptococcus mutans are members of the indigenous oral biota that colonize in the human oral cavity. S. mutans is widely known as a predominant bacterial pathogen involved in caries development (Loesche, 1986; Tanzer et al., 2001). S. sanguinis is one of the pioneer colonizers of oral cavity and may associate with aggregation and maturation of dental plaque (Rosan and Lamont, 2000). Studies also suggest that S. sanguinis is among the most prevalent streptococci in the saliva and dental plaque, especially on tooth surfaces with no caries (Becker et al., 2002; Carlsson et al., 1970; Loesche et al., 1975; Rosan and Lamont, 2000; Van Houte et al., 1970).

Previously, our laboratory conducted extensive studies on the natural histories of S. sanguinis and S. mutans infection in children (Caufield et al., 1993; Caufield et al., 2000; Li and Caufield, 1995; Li and Caufield, 1998; Li et al., 2005a). Based on observation of bacterial cultivation, we reported that S. sanguinis can be detected in an infant's oral cavity at about 9 months of age; and early colonization of S. sanguinis significantly delays the initial acquisition of S. mutans (Caufield et al., 2000). The presence of S. sanguinis, therefore, may serve as a protective or antagonistic role against colonization of S. mutans and other cariogenic bacteria. It has been observed that a greater S. mutans to S. sanguinis ratio was correlated with more caries, suggesting that S. mutans to S. sanguinis ratio may serve as an indicator for high risk for caries (De Stoppelaar et al., 1969; Loesche et al., 1975; Loesche and Bhat, 1976).

Currently, a number of PCR-based methods have been developed to identify S. mutans (Aguilera Galaviz L A, 2002; Arakawa et al., 2004; Chen et al., 2007; Hoshino T, 2004) or S. sanguinis (Li et al., 2003; Rudney et al., 1992; Rudney and Larson, 1999) in various samples, mostly are qualitative diagnostic assays, which may not suitable for microbiological evaluation of caries susceptibility. In contrast, real-time quantitative PCR with species-specific primers are capable to accurately detect and monitor colonization of target individual bacterial species in the saliva and dental plaque. Our research group has recently developed and tested species-specific primers for S. sanguinis and S. mutans in both pure culture and clinical samples (Chen et al., 2007; Li et al., 2003) with a high degree of sensitivity, specificity and reproducibility for quantification of the two microorganism.

Here we further evaluate the diagnostic application of these primers in longitudinal collected clinical samples of 0-3 year-old children. The purpose of this investigation was to validate this real-time quantitative PCR assay, by comparing the results with conventional cultural methods, in identifying and quantifying S. mutans and S. sanguinis. Our long-term goal is to develop a both sensitive and specific quantitative PCR assay that can be performed within minimal sample processing, independent of bacterial count from patients, with low assay variability and in a high throughout format.

### Materials & Methods

### Saliva Sample Collection and Enumeration by Culture Method

A total of 109 children were randomly selected from a Birmingham mother-child cohort study (Caufield et al., 2000; Ku et al., 2004; Li and Caufield, 1995; Li et al., 2005a). The level of S. mutans and S. sanguinis in the saliva of these children from new born to 36-month-old was previously determined by the enumeration of colony-forming units (CFU) on selective medium, mitis salivarius with bacitracin (MSB for S. mutans) or the non-selective MM10 sucrose blood agar plates (for S. sanguinis), as described previously (Caufield et al., 2000; Ku et al., 2004; Li and Caufield, 1995; Li et al., 2005a).

### Bacterial Genomic DNA Isolations

All colonies on MM10 plates were collected by sterile swabs and stored in skim milk storage medium at −70° C. until further analysis. In this study, the bacterial samples were dissolved at 4° C. The procedures of sample collection and DNA isolation have previously been published elsewhere(Li et al., 2005a; Li et al., 2005b). Briefly, total genomic DNA of each bacterial sample from MM10 medium was isolated by using MasterPure™ complete DNA Purification kit (Epicentre, Madison, Wis., WI, USA) following a phenol/chloroform/isoamyl alcohol extraction. Purified DNA was dissolved in TE buffer (10 mM Tris-HCl buffer, 1 mM EDTA, pH8.0). Quality and concentration of all DNA samples were determined by recording a UV spectrophotometer at 260 nm and 280 nm (DU 640 spectrophotometer, Beckman Instruments, Inc., Fullerton, Calif., USA). The DNA concentrations were adjusted to 10 μg/ml for future use.

Genomic DNAs of S. mutans UA159 and S. sanguinis 10556 strains were isolated from overnight cultures grown in Todd-Hewitt broth at 37° C. and were then extracted and purified as described previously (Chen et al., 2007; Li et al., 2003). The concentration of these DNA for standards were also adjusted to 10 μg/ml in water and then six additional serial dilutions (1:10) were made.

Species-Specific Primers for S. mutans and S. sanguinis

S. mutans PCR amplification was performed with primers Sm479F (5′-TCG CGA AAA AGA TAA ACA AAC A-3′ (SEQ ID NO:1)) and Sm479R (5′-GCC CCT TCA CAG TTG GTT AG-3′ (SEQ ID NO:2)) designed from a 14 kb Hae-III fragment. Amplification was performed using the same cycling condition as described previously (Chen et al., 2007). For the detection of S. sanguinis, the primers Sa475F (5′-GAA GCC ATT TTG CCT AGA TTG ATG G-3′(SEQ ID NO:18)) and Sa475R (5′-CCA TAC CGA TTC CTT ACT CTA AAT TT-3′ (SEQ ID NO:19)) were designed to amplify a 475 bp segment (Li et al., 2003).

### Optimization of Real-Time PCR Assay

To find the optimized condition of the primer concentration, the method of chessboard procedure was performed in this study (Rory Gunson, 2003). Briefly, this method requires a range of forward primer concentrations to be run against a range of reverse primer concentrations in the form of a matrix or chessboard. In this study, forward primers and reverse primers are tested between the concentrations of 0.2 μM and 1.0 μM. Annealing temperatures of these two sets of primers are determined experimentally by gradient temperature every 1° C. from 55° C. to 65° C. 100 fg and 10 fg of corresponding reference genomic DNAs are used as template to detect the efficiency and primer-dimer formation. A suitable annealing temperature is highly efficient without primer-dimers.

### Real-Time PCR Protocol

Real-time PCR was performed by the Opticon Monitor 2 (MJ Research Inc., Alameda, Calif.) using low profile 96-well polypropylene microplate (MJ Research Inc). Serial dilution of UA159 or ATCC 10556 genomic DNA was used as an external DNA standard for absolute quantification of either S. mutans or S. sanguinis amounts in clinical samples. Each reaction tube contained 25 μl of reaction mixture, including 1× QuantiTect SYBR Green PCR Master Mix (Qiagen Inc., Valencia, Calif.), and 0.4 μM of each pair of primers for S. mutans or S. sanguinis, separately. The template DNA was genomic DNA either from saliva (sample) or UA159 (standard of S. mutans), 10556 (standard of S. sanguinis). Each run contained a series of standards (with 10 ng, 1 ng, 100 pg, 10 pg, 1 pg, 100 fg, and 10 fg of standard DNA as template) and the unknowns (105 ng of total DNAs extracted from MM10). Each experiment included an NTC (No Template control), containing all the components of the reaction except for the template, which enables detection of contamination. The cycling conditions were 15 min at 95° C. for activation of HotStarTaq DNA Polymerase, 45 cycles of 15 s at 94° C. for denaturation, 30 s for annealing and 30 s at 72° C. for extension. The PCR reactions were subjected to a melting curve protocol present in the Opticon 2 software. Following the final cycle of the PCR, the reactions were heat denatured over a 30° C. temperature gradient at 0.5° C./s from 60 to 90° C.

PCR reactions were carried out in at least duplicate for each sample, triplicate if necessary. When these duplicates were in accordance, the sample was rated either negative or positive. Otherwise, with one positive result out of two, the sample was rated according to the third result. When calculation of streptococcal genome DNA is concerned, quantitative data will be taken into consideration only when the C (T) value of clinical specimen is among the range of standard curve. The raw data of real-time PCR indicated that pico-gram (pg) of target DNA were present in each PCR tube. Data were analyzed using Opticon Monitor 2 software (MJ Research Inc). The analytical specificity of the fluorescence signal was determined on the basis of subsequent melting curve analysis which gave a melting peak at 78° C. for individual target DNA. Furthermore, a 1.5% agarose gel electrophoresis was performed to correlate product length with melting peaks and identify product and primer-dimer bands. To rule out a false-positive result of the PCR amplification, 10% of the PCR products were randomly selected for sequence analysis.

### DNA Sequencing Analysis

PCR mixtures of these two pairs of primers were randomly chosen and sequenced as previously described (Chen et al., 2007). Briefly, PCR products are purified (Qiaquick PCR purification kit, Qiagen), and further sequenced by using an ABI model 3730 DNA sequencer (Applied Biosystems) in both directions. Nucleotide and deduced amino-acid sequences were analyzed with Chromas version 1.45 (Conor McCarthy, School of Health Science, Griffith University, Australia). A sequence similarity search of the nonredundant GenBank database was performed by using Nucleotide-nucleotide BLAST (blastn) program (National Center for Biotechnology Information, National Library of Medicine, National Institutes of Health, Bethesda, Md.) (Altschul et al., 1997) and sequences were aligned by using ClustalW program (Chema, 2003).

### Statistical Analysis

In order to compare the real-time PCR assay with conventional culture assays. Pearson Chi-Square and Fisher's Exact tests were calculated with the statistical software SPSS v.13.0 (SPSS, 2004).

### Results

Previously, the use of cultivation assay was considered the gold standard for the diagnosis of S. mutans or S. sanguinis colonization. The comparison of the PCR assay and cultivation assay in detection of S. mutans and S. sanguinis are summarized in TABLE 9. Overall, the concordance between the two methods was 91.8% and 75.4% for S. mutans and S. sanguinis, respectively. PCR assay demonstrated high specificity compared with the culture methods and significantly improved the positive detection rate for either S. mutans (p<0.05) or S. sanguinis (p<0.05).

TABLE 9
Comparisons of the PCR assay and cultivation assay in detection
of S. mutans (A) and S. sanguinis (B) in the saliva of young children.

| | Real-time qPCR | | |
|---|---|---|---|
| Culture | No. of positive (%) | No. of negative (%) | Total (%) |

A. S. mutans

| | | | |
|---|---|---|---|
| No. of positive | 42 (79.2) | 11 (20.8) | 53 (9.1) |
| No. of negative | 37 (7.0) | 494 (93.0) | 531 (90.9) |
| Sum | 79 (13.5) | 505 (86.5) | 584 (100) |

Concordance = (42 + 494)/584 * 100% = 91.8%.
Sensitivity = 42/79 * 100% = 53.2%
Specificity = 494/505 * 100% = 97.8%

B. S. sanguinis

| | | | |
|---|---|---|---|
| No. of positive | 53 (75.7) | 17 (24.3) | 70 (12.0) |
| No. of negative | 127 (24.7) | 387 (75.3) | 514 (88.0) |
| Sum | 180 (30.8) | 404 (69.2) | 584 (100) |

Concordance = (53 + 387)/584 * 100% = 75.4%.
Sensitivity = 53/180 * 100% = 29.4%
Specificity = 387/404 * 100% = 95.8%

Optimization of the real-time qPCR using the chessboard of primer concentration showed that 0.4 μM of each primer for S. mutans or S. sanguinis was a suitable primer concentration for applying real-time qPCR assay in clinical samples. Annealing temperature of 56° C. for S. mutans or 60° C. for S. sanguinis was highly efficient in amplification without formation of primer-dimmer.

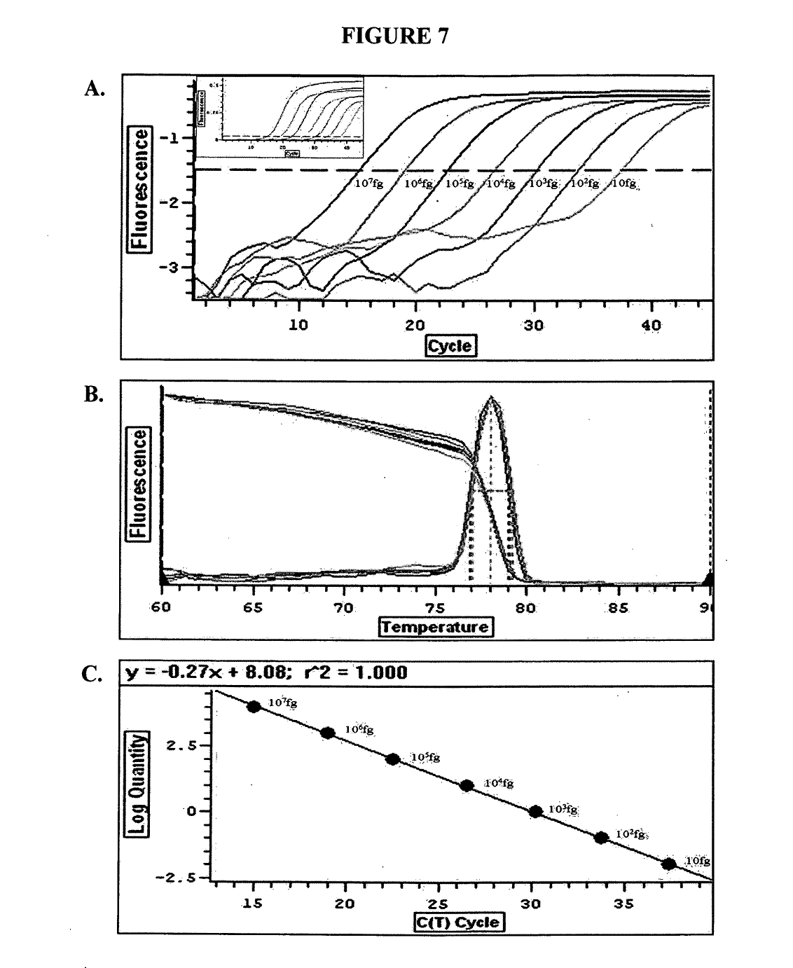

A typical plot of data generated by real-time qPCR using SYBR Green I detection kit was illustrated in FIG. 7. The PCR signal was initially below the limit of detection and increases with cycle number to cross a threshold. The threshold cycle C(T)s served as a tool for calculation of the starting template amount in each sample and increased in fluorescence (FIG. 7A). A set standard samples with known template amounts was used for generating a standard curve. The C(T)s were plotted against the log of the template amount, resulting in a straight line (FIG. 7B). Thus, C(T) values for these samples and the standard curve are then used to calculate the amount of starting template in the experimental samples. The melting curve analysis following the final cycle of the PCR demonstrated that dissociation of the PCR reactions consistently produced a single peak for S. mutans-specific (FIG. 7C), indicating the presence of only one product in the reaction without primer-dimmer. All PCR results were further verified by 1.0% agarose gel electrophoresis.

The sensitivity and dynamic range of the real-time qPCR assays were determined by amplifying serial dilutions of genomic DNA of corresponding reference strain. The lower limit of detection was defined as the point at which the relationship between C(T) cycle and log quantity became nonlinear. Serial dilutions of UA159 (S. mutans) or 10556 (S. sanguinis) genomic DNA (10 ng to 10 fg) were amplified. The standard curves for calibration of either the S. mutans or the S. sanguinis assays showed a perfect linearity using qPCR data over a 7-log dynamic range of 10 ng to 10 fg. The limit of detection level was 10 fg of the genomic DNA for either S. mutans or S. sanguinis pure culture. Approximately 10% of PCR products of clinical samples were random selected for DNA sequence analysis. The results further confirmed the specificity of these primers, with 97%-100% identity of S. mutans reference strain UA159 (GenBank accession number AE014133) and 98%-100% of S. sanguinis ATCC 10556 (GenBank accession number AY277586).

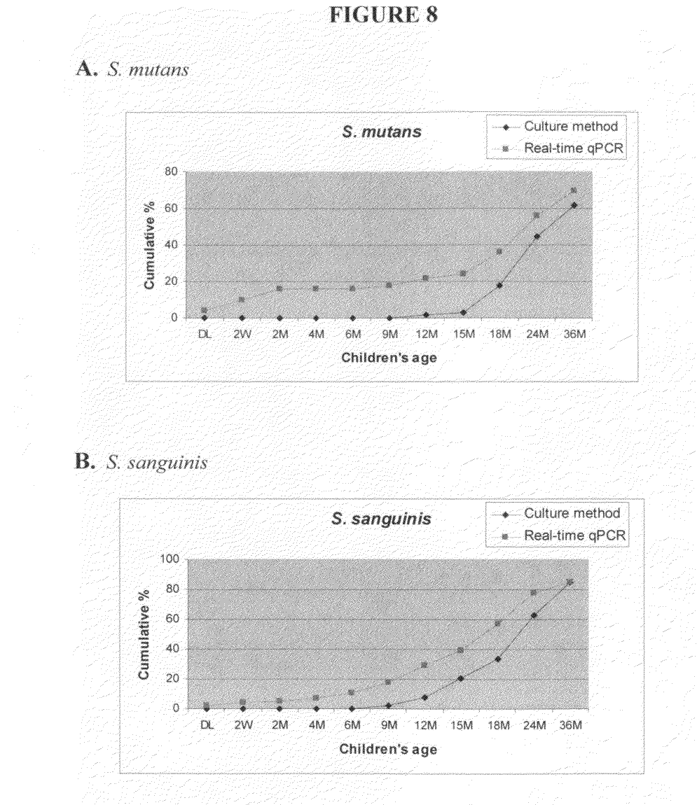

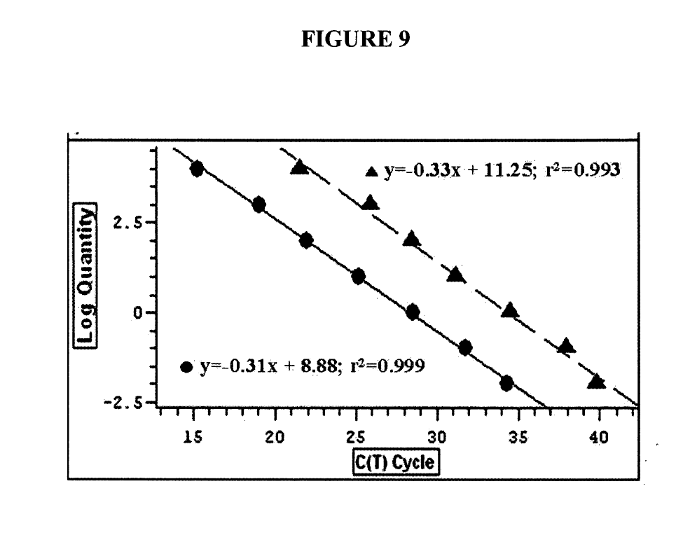

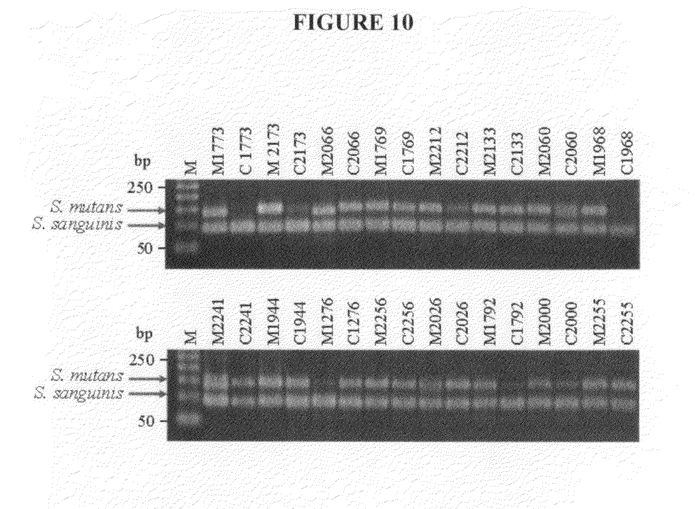

Interestingly, S. mutans was detected successfully by PCR assay from 3.6% children at birth to 36.8% at 36 months. In comparison, previously using conventional culture assay, we were able to detect S. mutans in 1.8% of children at birth and 54.7% at 36 months old. The cumulative percentage data show a higher detection rate in every age group from real-time qPCR than the culture method (FIG. 8A). The similar results were observed for S. sanguinis. PCR assay showed S. sanguinis positive in 7.3% of new born babies and 68.4% in 36-month-old children; the percentage was consistently higher for PCR than the culture assay at each age group (FIG. 8B). We also observed a positive correlation between the age and the prevalence of S. mutans (P<0.001, Non-parametric test) and prevalence of S. sanguinis (P<0.001, Non-parametric test) for PCR assay.

### Discussion

In the present study, the PCR assay proved to be more sensitive than culture method in detecting S. mutans and S. sanguinis colonization in children's saliva. The initial colonization of these two microorganisms appears to be earlier than we previously reported based on the Birmingham mother-child cohort survey. The major findings of our previous study included that the initial acquisition of S. mutans occurred at the median age of 26 months (Caufield et al., 1993); while the colonization of the S. sanguinis occurs during a discrete “window of infectivity” at a median age of 9 months in the infants (Caufield et al., 2000). Furthermore, early colonization of S. sanguinis and its elevated levels in the oral cavity were correlated with delayed colonization of cariogenic S. mutans and, therefore, may lower rates of tooth decay in children (Caufield et al., 2000; Ku et al., 2004; Li and Caufield, 1995). The use of saliva for diagnostic purposes has been the subject of considerable research (Mandel, 1990). Saliva is easy to obtain and contains bacteria from different oral sites including oral mucosal sites and supra- and sub-gingival plaque from the teeth, when present. Salivary microbiological diagnosis provides a noninvasive, inexpensive, and rapid technique for the detection and quantification of oral pathogens. Consequently, studies have evaluated the presence and levels of bacteria in saliva in relation to the caries status. Our previous study used the culture technique to investigate the presence of S. mutans and S. sanguinis in unstimulated saliva samples and show both bacteria have the window of infectivity at different age. Wan group (Wan et al., 2001a; Wan et al., 2001b; Wan et al., 2003) found that 30% infants had S. mutans in their saliva with culture method. In contrast, the polymerase chain reaction (PCR) technique can detect S. mutans in stimulated whole saliva with the prevalence of 93.3% in 20-year-old young adults (Oho et al., 2000) and 100% in unstimulated saliva of 10 healthy 3-7 year-old children (Hoshino T, 2004).

Real-time PCR is a relatively new molecular technique to detect and quantify caries related pathogens and has been used for oral samples by several authors (Hata et al., 2006; Price et al., 2007; Yano et al., 2002; Yoshida et al., 2003). Detection chemistries in real-time quantitative PCR today can be either probe- or non-probe based (Wilhelm and Pingoud, 2003). The widely used non-probe-based chemistry detects the binding of SYBR Green I to ds (double-stranded) DNA. In solution, the unbound dye exhibits little fluorescence; during the PCR assay, increasing amounts of dye bind to the nascent ds DNA. When monitored in real-time, this results in an increase in the fluorescence signal as the polymerization proceeds. The sensitivity of this technique allows the detection of very small numbers of pathogens, one single copy of cell in theory. The PCR product can be verified by plotting fluorescence as a function of temperature to generate a melting curve of the amplicon. An important advantage of non-probe-based chemistries is that in most instances optimized conventional RT-PCR assays can be converted immediately into real-time assays, while their specificity remains dependent on the specificity of the primers (Stephen A. Bustin, 2005; Wilhelm and Pingoud, 2003; Zhang and Fang, 2006). Previous studies have determined that a large qPCR target sequence is more appropriate for DNA quantifications than is a short qPCR target (Swango et al., 2006; Timken et al., 2005), which may indicate that target sequences around 500 bp in this experiment are suitable in real-time qPCR compared to probe-based Taqman assay, in which the maximum target size is 150 bp. The combination of excellent sensitivity and specificity, low contamination risk, ease of performance and speed in a closed-tube format, has made real-time PCR technology an appealing alternative to conventional culture-based or immunoassay-based testing methods used in the clinical microbiology for diagnosing many infectious diseases (Espy et al., 2006).

The results from this study have shown that the tested real-time quantitative PCR assay is able to identify and quantify S. mutans and S. sanguinis, with a high degree of sensitivity and specificity when compared with standard culturing techniques. The genome size of S. mutans UA159 is reported to be 2,030,921 base pairs (GenBank accession no, NC_{—}004350) (Ajdic et al., 2002), which means 10 fg of UA159 genomic DNA as low as detection level corresponds to 4.6 copy. Although the genomic size of S. sanguinis 10556 has not published yet, the complete genome size of S. sanguinis SK36 is newly reported to be 2,388,435 base pairs (GenBank accession no, NC_{—}009009) (Xu et al., 2007). The genomic size of 10556 is predicted to be close or similar to SK36 since they belong to the same species, which means 10 fg of 10556 genomic DNA as detection level corresponds to 3.9 copy.

When tested for the detection of S. mutans, real-time quantitative PCR yielded a prevalence of 8.5% in this study. The evaluation of S. mutans prevalence results in preschool children using PCR technology is limited and gives very heterogeneous results when assessing the scientific literature, from very high prevalence (100%) of edentulous children over 4-7 months old in Brazil (Klein et al., 2004) and 72.8% in Japanese pre-school children (Okada et al., 2002), to relatively low prevalence reported (38.7%) in 1.5 to 7 years old twins in Brazil (Corby et al., 2005), and (less than 48%) in 3-4 years old Japanese children (Hata et al., 2006; Seki et al., 2006); eventually, variance between 32% and 74% of 3-7 years old in Mexico occurred in means of different PCR primers (Aguilera Galaviz L A, 2002). The explanations for this heterogeneity may vary in terms of children age, caries experience, sample size, methodological issues, different bacterial serotypes and true geographical differences, although large ecological studies in specific populations around the world using standardized highly specific quantitative PCR should be performed in order to understand the true prevalence and geographical distribution of this bacterial species.

Comparing the culture and PCR results, a significant diagnostic agreement was found, especially when considering all groups of children together. In 18 cases, PCR yielded a positive result, associated with a negative by culture. This could be explained by the lower limit of detection and the stringent growing conditions required by culture. Conversely, 22 samples were positive by culture and negative for PCR, most of them belong to ≧24-month sample periods. Considering that CFU counting by culture method was determined by the colony on MSB medium, which is a specific selective medium for S. mutans, while DNA as template for real-time qPCR were isolated and purified from MM10 nonselective medium, the competition among varied species for nutrition and space on MM10, and significant escalation in S. mutans counts and change in microbial community in the oral cavity of older children may explain this observation. Another possibility is the presence of false negatives with PCR. This could be justified by the presence of PCR inhibitors in total DNA samples and genetic variations in the partial sequence of serine protease gene (htrA) used as a primer for this PCR assay.

S. sanguinis was detected in 31.2% of the children using PCR and in 12% using culture. There is a significant difference in sensitivity between these two methods. Overall, in 127 cases a positive PCR result was associated with a negative culture, and the opposite was true in seventeen cases. These 127 cases can be easily explained by the lower limit of detection and the stringent growing conditions required by culture. Our results are lower than what has been reported as a high prevalence (over 98.5%) of S. sanguinis in 1.5 to 7 years old twins in Brazil by PCR-based method (Corby et al., 2005), which may due to age group, primers, and demographic variance.

In conclusion, we have established a sensitive and relatively simple method for PCR amplification of S. mutans and S. sanguinis from clinical samples. These results demonstrated the feasibility and clinical applicability of using quantitative real-time PCR assay for determining and monitoring S. mutans and S. sanguinis colonization in the oral cavity.

### REFERENCES

Aguilera Galaviz L A AMMC, Estrada Garcia I C. (2002). Detection of potentially cariogenic strains of Streptococcus mutans using the polymerase chain reaction. J Clin Pediatr Dent. 27(1):47-51.
Ajdic D, McShan W M, McLaughlin R E, Savic G, Chang J, Carson M B, Primeaux C, Tian R, Kenton S, Jia H, Lin S, Qian Y, Li S, Zhu H, Najar F, Lai H, White J, Roe B A, Ferretti J J (2002). Genome sequence of Streptococcus mutans UA159, a cariogenic dental pathogen. Proc Natl Acad Sci U S A 99(22):14434-9.
Altschul S F, Madden T L, Schaffer A A, Zhang J, Zhang Z, Miller W, Lipman DJ (1997). Gapped BLAST and PSI-BLAST: a new generation of protein database search programs. Nucleic Acids Res 25(17):3389-402.
Arakawa H, Karasawa K, Igarashi T, Suzuki S, Goto N, Maeda M (2004). Detection of cariogenic bacteria genes by a combination of allele-specific polymerase chain reactions and a novel bioluminescent pyrophosphate assay. Anal Biochem 333(2):296-302.
Becker M R, Paster B J, Leys E J, Moeschberger M L, Kenyon S G, Galvin J L, Boches S K, Dewhirst F E, Griffen A L (2002). Molecular analysis of bacterial species associated with childhood caries. J Clin Microbiol 40(3):1001-9.
Carlsson J, Grahnen H, Jonsson G, Wikner S (1970). Establishment of Streptococcus sanguis in the mouths of infants. Arch Oral Biol 15(12):1143-8.
Caufield P W, Cutter G R, Dasanayake A P (1993). Initial acquisition of mutans streptococci by infants: evidence for a discrete window of infectivity. J Dent Res 72(1):37-45.
Caufield P W, Dasanayake A P, Li Y, Pan Y, Hsu J, Hardin J M (2000). Natural history of Streptococcus sanguinis in the oral cavity of infants: evidence for a discrete window of infectivity. Infect Immun 68(7):4018-23.
Chen Z, Saxena D, Caufield P W, Ge Y, M. W, Li Y (2007). Development of species-specific primers for detection of Streptococcus mutans in mixed bacterial samples. FEMS Microbiol Lett 272(154-162.
Chema R, Sugawara, Hideaki, Koike, Tadashi, Lopez, Rodrigo, Gibson, Toby J, Higgins, Desmond G, Thompson, Julie D. (2003). Multiple sequence alignment with the Clustal series of programs. Nucleic Acids Res 31(13):3497-500.
Corby P M, Lyons-Weiler J, Bretz W A, Hart T C, Aas J A, Boumenna T, Goss J, Corby A L, Junior H M, Weyant R J, Paster B J (2005). Microbial risk indicators of early childhood caries. J Clin Microbiol 43(11):5753-9.
De Stoppelaar J D, Van Houte J, Backer Dirks 0 (1969). The relationship between extracellular polysaccharide-producing streptococci and smooth surface caries in 13-year-old children. Caries Res 3(2):190-9.
Espy M J, Uhl J R, Sloan L M, Buckwalter S P, Jones M F, Vetter E A, Yao J D, Wengenack N L, Rosenblatt J E, Cockerill F R, 3rd, Smith T F (2006). Real-time PCR in clinical microbiology: applications for routine laboratory testing. Clin Microbiol Rev 19(1):165-256.
Hata S, Hata H, Miyasawa-Hori H, Kudo A, Mayanagi H (2006). Quantitative detection of Streptococcus mutans in the dental plaque of Japanese preschool children by real-time PCR. Lett Appl Microbiol 42(2):127-31.
Hoshino T K M, Shimizu N, Hoshino N, Ooshima T, Fujiwara T. (2004). PCR detection and identification of oral streptococci in saliva samples using gtf genes. Diagn Microbiol Infect Dis. 48(3):195-9.
Klein M I, Florio F M, Pereira A C, Hofling J F, Goncalves R B (2004). Longitudinal study of transmission, diversity, and stability of Streptococcus mutans and Streptococcus sobrinus genotypes in Brazilian nursery children. J Clin Microbiol 42(10):4620-6.
Ku C, Y L, P W C, D S, Z P (2004). Analysis of Oral Bacterial Diversity Using PCR-Denaturing Gradient Gel Electrophoresis. J Dent. Res. 83. Li Y, Caufield P W (1995). The fidelity of initial acquisition of mutans streptococci by infants from their mothers. J. Dent Res 74(2):681-5.
Li Y, Caufield P W (1998). Arbitrarily primed polymerase chain reaction fingerprinting for the genotypic identification of mutans streptococci from humans. Oral Microbiol Immunol 13(1):17-22.
Li Y, Pan Y, Qi F, Caufield P W (2003). Identification of Streptococcus sanguinis with a PCR-generated species-specific DNA probe. J Clin Microbiol 41(8):3481-6.
Li Y, Caufield P W, Dasanayake A P, Wiener H W, Vermund S H (2005a). Mode of delivery and other maternal factors influence the acquisition of Streptococcus mutans in infants. J Dent Res 84(9):806-11.
Li Y, Ku C Y, Xu J, Saxena D, Caufield P W (2005b). Survey of oral microbial diversity using PCR-based denaturing gradient gel electrophoresis. J Dent Res 84(6):559-64.
Loesche W J, Rowan J, Straffon L H, Loos P J (1975). Association of Streptococcus mutants with human dental decay. Infect Immun 11 (6):1252-60.
Loesche W J, Bhat M (1976). Evaluation of diagnostic broths for Streptococcus mutans. In: Microbiol aspects of dental caries. H M Stiles, W J Loesche and O'Brien editors. Washington, D.C.: Informational Retrieval Inc., pp. 291-301.
Loesche W J (1986). Role of Streptococcus mutans in human dental decay. Microbiol. Rev 50(4):353-80.
Mandel I D (1990). The diagnostic uses of saliva. J Oral Pathol Med 19(3):119-25.
Oho T, Yamashita Y, Shimazaki Y, Kushiyama M, Koga T (2000). Simple and rapid detection of Streptococcus mutans and Streptococcus sobrinus in human saliva by polymerase chain reaction. Oral Microbiol Immunol 15(4):258-62.
Okada M, Soda Y, Hayashi F, Doi T, Suzuki J, Miura K, Kozai K (2002). PCR detection of Streptococcus mutans and S. sobrinus in dental plaque samples from Japanese pre-school children. J Med Microbiol 51(5):443-7.
Price R R, Viscount H B, Stanley M C, Leung K P (2007). Targeted profiling of oral bacteria in human saliva and in vitro biofilms with quantitative real-time PCR. Biofouling 23(3):203-13.
Rory Gunson GGaWFC (2003). Optimisation of PCR reactions using primer chessboarding. Journal of Clinical Virology 26(369-373. Rosan B, Lamont R J (2000). Dental plaque formation. Microbes Infect 2(13):1599-607.
Rudney J D, Neuvar E K, Soberay A H (1992). Restriction endonuclease-fragment polymorphisms of oral viridans streptococci, compared by conventional and field-inversion gel electrophoresis. J Dent Res 71(5):1182-8.
Rudney J D, Larson C J (1999). Identification of oral mitis group streptococci by arbitrarily primed polymerase chain reaction. Oral Microbiol Immunol 14(1):33-42.
Seki M, Yamashita Y, Shibata Y, Torigoe H, Tsuda H, Maeno M (2006). Effect of mixed mutans streptococci colonization on caries development. Oral Microbiol Immunol 21(1):47-52.
Stephen A. Bustin R M (2005). Real-time reverse transcription PCR (qRT-PCR) and its potential use in clinical diagnosis clinical Science 109(365-379.
Swango K L, Timken M D, Chong M D, Buoncristiani M R (2006). A quantitative PCR assay for the assessment of DNA degradation in forensic samples. Forensic Sci Int 158(1):14-26.
Tanzer J M, Livingston J, Thompson A M (2001). The microbiology of primary dental caries in humans. J Dent Educ 65(10):1028-37.
Timken M D, Swango K L, Orrego C, Buoncristiani M R (2005). A duplex real-time qPCR assay for the quantification of human nuclear and mitochondrial DNA in forensic samples: implications for quantifying DNA in degraded samples. J Forensic Sci 50(5):1044-60.
Van Houte J, Gibbons R J, Banghart S B (1970). Adherence as a determinant of the presence of Streptococcus salivarius and Streptococcus sanguis on the human tooth surface. Arch Oral Biol 15(11):1025-34.
Wan A K, Seow W K, Purdie D M, Bird P S, Walsh L J, Tudehope D I (2001a). Oral colonization of Streptococcus mutans in six-month-old predentate infants. J Dent Res 80(12):2060-5.
Wan A K, Seow W K, Walsh L J, Bird P, Tudehope D L, Purdie D M (2001b). Association of Streptococcus mutans infection and oral developmental nodules in pre-dentate infants. J Dent Res 80(10):1945-8.
Wan A K, Seow W K, Purdie D M, Bird P S, Walsh L J, Tudehope D I (2003). A longitudinal study of Streptococcus mutans colonization in infants after tooth eruption. J Dent Res 82(7):504-8. Wilhelm J, Pingoud A (2003). Real-time polymerase chain reaction. Chembiochem 4(11):1120-8.
Xu P, Alves J M, Kitten T, Brown A, Chen Z, Ozaki L S, Manque P, Ge X, Serrano M G, Puiu D, Hendricks S, Wang Y, Chaplin M D, Akan D, Paik S, Peterson D L, Macrina F L, Buck G A (2007). Genome of the opportunistic pathogen Streptococcus sanguinis. J Bacteriol 189(8):3166-75.
Yano A, Kaneko N, Ida H, Yamaguchi T, Hanada N (2002). Real-time PCR for quantification of Streptococcus mutans. FEMS Microbiol Lett 217(1):23-30.
Yoshida A, Suzuki N, Nakano Y, Kawada M, Oho T, Koga T (2003). Development of a 5′ nuclease-based real-time PCR assay for quantitative detection of cariogenic dental pathogens Streptococcus mutans and Streptococcus sobrinus. J Clin Microbiol 41(9):4438-41.
Zhang T, Fang H H (2006). Applications of real-time polymerase chain reaction for quantification of microorganisms in environmental samples. Appl Microbiol Biotechnol 70(3):281-9.

### Example 4

Streptococcus mutans is the major microbial pathogen associated with dental caries. Streptococcus sanguinis is known to play an antagonistic role against the colonization of cariogenic bacterium S. mutans in the oral cavity. Based on conventional culture methods, it has been suggested that S. mutans/S. sanguinis ratio may serve as a caries risk indicator.

Dental caries is an infectious disease caused by multivariable factors including the presence of fermentable sugars, susceptibility of the tooth, and the presence of cariogenic bacteria in the oral cavity. A predominant bacterial pathogen involved is thought to be Streptococcus mutans.

Streptococcus sanguinis is a key member of the indigenous oral biota and one of the most prevalent members of the oral streptococci, especially on tooth surfaces with no caries present. Previous studies have showed that not only the amount of virulent S. mutans correlated with the caries risk of a child, but the ratio of S. mutans to S. sanguinis is also a concern. A higher ratio of S. mutans to S. sanguinis is reported to be associated with caries. Thus being able to accurately assess the relative levels of these two bacteria in the oral biota is important.

Traditionally, methods used for detecting and quantifying bacteria in the oral cavity primarily relied on cultivation via selective media. This may not reflect the actual amount of bacteria in situ nor accurately quantify multiple bacterial species at the same time.

Currently, PCR-based techniques have developed that rapidly and accurately detect and quantify DNA of S. mutans and S. sanguinis in clinical samples by using species-specific designed primers. Our lab has also developed a duplex real-time PCR assay, which can simultaneously detect levels of both bacterial DNA in one test tube using species-specific primers and probes.

The goal of this study was to analyze the specificity and sensitivity of this new method; to detect and quantify S. mutans and S. sanguinis in the saliva of mother-child pairs, including caries-free and caries-active children; and to evaluate the association between S. mutans/S. sanguinis ratio and caries status.

Objectives. To detect and quantify S. mutans and S. sanguinis in saliva of a mother-child cohort using duplex real-time PCR (qPCR) and to evaluate the association of S. mutans/S. sanguinis ratio and caries status.

Methods. Twenty 2 to 8-year-old children (10 caries active vs. 10 caries free) and their mothers were included in this study (see Table 10). Stimulated whole salivary samples were collected and total bacterial genomic DNA was isolated. A duplex real-time PCR assay with species-specific primers and probes were used for this study. The primers were as set out in Tables 4 and 5 in Example 2 above.

TABLE 10
Gender and Age Distribution in the 20 Mother-Child Pairs

| | | | Age (yr) | |
|---|---|---|---|---|
| Group | N | Gender | (Mean ± SD) | Range |
| Mother | 20 | 20 (100%) | 31.6 ± 5.5 | 22.5-42.8 |
| Child | 20 | Boy: 9 (45%) | 5.6 ± 1.9 | 2.4-8.3 |
| | | Girl: 11 (55%) | | |

TABLE 11
Caries Prevalence and Mean Caries Score*

| Group | Caries Presence | Mean ± SD | Range |
|---|---|---|---|
| Mother | 20 (100%) | 51.9 ± 27.5 | 14-107 |
| Child | 10 (50%) | 9.6 ± 7.4 | 1-27 |

*mean caries score was the sum decayed, missing, and filled tooth surfaces

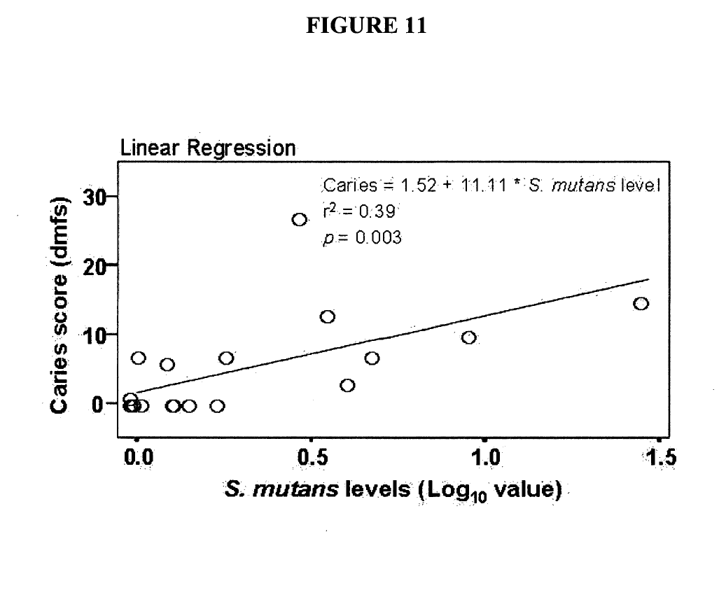

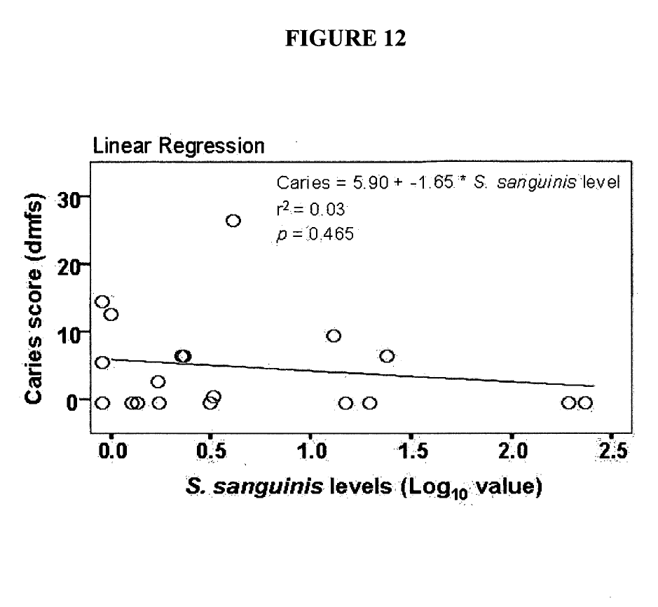

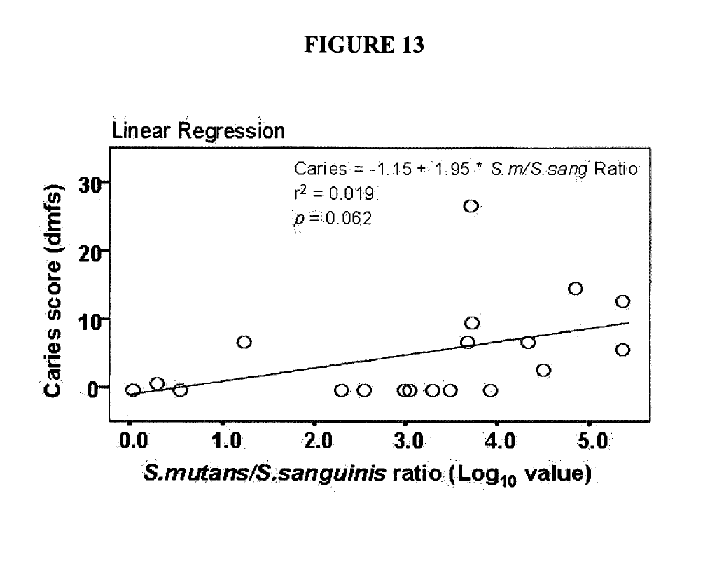

Results. Using fluorescent Taqman assay, S. mutans and S. sanguinis were detected in the saliva of 95% and 100% of the children, respectively. The mean level of S. mutans was significantly higher in caries-active children than caries-free children (p=0.015) (Table 12 and FIG. 11). There was a 10-fold increase in S. sanguinis level in caries-free children compared to caries-active children, though the difference was not statistically significant given the small sample size (FIG. 12). The S. mutans/S. sanguinis ratio might be correlated with children's caries severity (p=0.062) (FIG. 13).

TABLE 12
Prevalence and mean values of S. mutans and S. sanguinis in the 20
mother-child pairs

| | Mother | Child | |
|---|---|---|---|
| | All caries Active | Caries Active | Caries Free |
| Microorganism | N = 19 | N = 10 | N = 10 |
| S. mutans | | | |
| Positive (%) | 19 (100%) | 10 (100%) | 9 (90%) |
| Mean levels | 3.6 ± 6.1 | 5.0 ± 8.6* | 0.2 ± 0.3* |
| (pg/100 mg total | | | |
| DNA) | | | |
| S. sanguinis | | | |
| Positive (%) | 18 (94.7%) | 10 (100%) | 10 (100%) |
| Mean levels | 35.7 ± 56.7 | 5.2 ± 8.0 | 52.3 ± 97.4 |
| (pg/100 mg total | | | |
| DNA) | | | |

*p-value = 0.015; Nonparametric tests for two independent means.

Conclusions. This study demonstrated significant correlations between caries severity and S. mutans in children. S. mutans/S. sanguinis ratio may serve as a meaningful indicator for identifying high risk children for caries.

The following summarizes the results of this study:

1). Both species-specific primers and probes were specific to the targeted bacteria, S. mutans or S. sanguinis. Using the duplex real-time qPCR assay, the minimum detective level was 100 fg of genomic DNA for either S. mutans or S. sanguinis in the mixed total bacterial samples.

2). The prevalence of S. mutans and S. sanguinis in the saliva of the children were 95% and 100%, respectively. Almost all the mothers were S. mutans and S. sanguinis positive as well.

3). A significantly higher mean level of S. mutans was found in caries-active children compared to that of caries free children (p=0.015). The result also revealed that S. mutans level had significant positive correlation with caries severity (r²=0.39; p=0.003).

4). Although a significant difference was not detectable due to the small sample size, a 10-fold increase in the level of S. sanguinis was found in the saliva of caries-free children compared to the caries-active children.

5. A correlation of p-value=0.062 was found for the ratio of S. mutans/S. sanguinis and caries severity, indicating that S. mutans/S. sanguinis ratio can serve as a clinical indicator for caries risk in children. Further study with larger sample size is being undertaken.

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

What is claimed is:

1. A method for determining the presence or amount of S. mutans in a sample or subject which comprises
(a) isolating nucleic acid from said sample or subject;
(b) amplifying Sm479F/Sm479R targeted S. mutans sequence or a portion thereof using PCR or other amplification technology; and
(c) determining the presence and amount of the Sm479F/Sm479R targeted PCR product sequence, thereby determining the presence or amount of S. mutans in said sample or subject.

2. A method for simultaneously determining the presence or amount of S. mutans and S. sanguinis in a sample or subject which comprises
(a) isolating nucleic acid from said sample or subject;
(b) amplifying both Sm479F/Sm479R targeted S. mutans sequence PCR fragment and SSA-2 targeted S. sanguinis sequence PCR fragment from said nucleic acid; and
(c) detecting and quantitating both of the amplified portion of Sm479F/Sm479R targeted S. mutans sequence and the amplified portion of SSA-2 targeted S. sanguinis sequence obtained in step (b), thereby determining the presence or amount of both S. mutans and S. sanguinis in said sample or subject.

3. The method of claim 2, wherein step (b) is performed using a set of primers, wherein said set of primers contains primer pair X and Y and primer pair A and B, wherein
(i) the X and Y primer pair are complementary to a portion of the Sm479F/Sm479R targeted S. mutans sequence;
(ii) the A and B primer pair are complementary to a portion of the SSA-2 targeted S. sanguinis sequence;
and both the sequence in between primers X and Y and the sequence in between primers A and B are amplified, thereby obtaining two distinct amplified fragments; and both of the amplified fragments obtained in step (c) are detected and quantified, thereby determining the presence or amount of S. mutans and S. sanguinis in said sample or subject.

4. The method of claim 3, wherein primer X has the sequence corresponding to SEQ ID NO: 3, or a fragment thereof which is at least ten bases long, primer Y has the sequence corresponding to SEQ ID NO: 4, or a fragment thereof which is at least ten bases long, primer A has the sequence corresponding to SEQ ID NO: 6, or a fragment thereof which is at least ten bases long, primer Y has the sequence corresponding to SEQ ID NO: 7.

5. The method of claim 3, wherein primer X has the sequence corresponding to SEQ ID NO: 1, or a fragment thereof which is at least ten bases long, and primer Y has the sequence corresponding to SEQ ID NO: 2, or a fragment thereof which is at least ten bases long.

6. The method of claim 1 wherein step (b) is performed in the presence of a fluorogenic probe; and wherein the amount of fluorescence in step (c) is indicative of the presence and amount of PCR product, thereby determining the presence or amount of S. mutans in said sample or subject.

7. The method of claim 2 wherein step (b) is performed in the presence of both an S. mutans product specific fluorogenic probe, and an S. sanguinis product specific fluorogenic probe, wherein the fluorogenic probes have distinct fluorophores; and wherein the amount of fluorescence of each fluorophore in step (c) is indicative of the presence and amount of PCR product, thereby determining the presence or amount of both S. mutans and S. sanguinis in said sample or subject.

8. The method of claim 6 which utilizes a set of Sm479F/Sm479R S. mutans primers selected from SEQ ID NO: 1 and 2 or SEQ ID NO: 3 and 4 and a fluorogenic probe sequence of SEQ ID NO: 5.

9. The method of claim 7 which utilizes a set of Sm479F/Sm479R S. mutans primers selected from SEQ ID NO: 1 and 2 or SEQ ID NO: 3 and 4 and a S. mutans fluorogenic probe sequence of SEQ ID NO: 5, and a set of SSA-2 S. sanguinis primers selected from SEQ ID NO: 6 and 7 or SEQ ID NO: 18 and 19 and a S. sanguinis fluorogenic probe sequence of SEQ ID NO: 8.

10. A test kit for determining the presence or amount of S. mutans and/or S. sanguinis in a sample or subject, comprising:
(a) a predetermined amount of a first PCR primer set which amplifies Sm479F/Sm479R targeted S. mutans sequence or a portion thereof;
(b) a predetermined amount of a second PCR primer set which amplifies SSA-2 targeted S. sanguinis sequence or a portion thereof;
(c) other reagents, optionally including a fluorogenic probe specific for the amplified Sm479F/Sm479R targeted S. mutans PCR product and a distinct fluorogenic probe specific for the amplified SSA-2 targeted S. sanguinis PCR product; and
(d) directions for use of said kit.

11. The test kit of claim 10, wherein the first PCR primer set has sequences corresponding to SEQ ID NO: 1 and SEQ ID NO: 2, or SEQ ID NO: 3 and SEQ ID NO:4, or fragments thereof which are at least ten bases long.

12. The test kit of claim 10, wherein the second PCR primer set has sequences corresponding to SEQ ID NO: 6 and SEQ ID NO: 7, or SEQ ID NO: 18 and 19, or fragments thereof which are at least ten bases long.

13. The test kit of claim 10, wherein the S. mutans Sm479F/Sm479R product fluorogenic probe has a sequence corresponding to SEQ ID NO: 5, or a fragment thereof which is at least ten bases long.

14. The test kit of claim 10, wherein the S. sanguinis SSA-2 product fluorogenic probe has a sequence corresponding to SEQ ID NO: 8, or a fragment thereof which is at least ten bases long.

15. An isolated oligonucleotide primer having a sequence selected from SEQ ID NO: 1, 2, 3, 4, 6 or 7, or a fragment thereof which is at least ten bases long.

16. A composition of a primer pair and probe set comprising the sequences SEQ ID NO: 3, 4 and 5 in combination suitable for amplification and detection of S. mutans in a sample.

17. A composition of a primer pair and probe set comprising the sequences SEQ ID NO: 6, 7 and 8 in combination suitable for amplification and detection of S. sanguinis in a sample.

18. A composition of primer pairs and probes in combination, suitable for simultaneous amplification and detection of S. mutans and S. sanguinis in a sample, comprising the sequences SEQ ID NO: 3, 4, 5, 6, 7 and 8.

19. The method or kit of claim 6, 7 or 10, wherein the fluorophore on the fluorogenic probe(s) is selected from 6-carboxyfluoroscein (FAM), 2′-chloro-7′-phenyl-1,4-dichloro-6-carboxyfluorescein (VIC), tetrachloro-6-carboxyfluorescein, and hexachloro-6-carboxyflorescein.

20. The method or kit of claim 6, 7 or 10, wherein the fluorogenic probe(s) further comprise a covalently attached quencher.
